(19) United States
(12) Patent Application Publication
(10) Pub. No.: US 2011/0067253 A1
(43) Pub. Date: Mar. 24, 2011

(54) Method and System for Detecting Parameters for the Characterization of Motion Sequences at the Human Body and Computer-Implemented Method for Analyzing Parameters for the Characterization of Motion Sequences at the Human Body

(76) Inventor: Tobias Happel, Berlin (DE)

(21) Appl. No.: 12/712,291

(22) Filed: Feb. 25, 2010

Related U.S. Application Data
(63) Continuation of application No. PCT/EP2009/006806, filed on Sep. 21, 2009.

(51) Int. Cl. A61B 5/103

(52) U.S. Cl. 33/512

(57) ABSTRACT

The invention relates to a system for detecting function parameters for the characterization of motion sequences at the human body, comprising: a bending sensor with at least one strain gauge for detecting bending parameters, wherein said at least one strain gauge changes its impedance during an extension or compression, and sensor electronics for reading out and processing the bending parameters detected by said at least one strain gauge; and a fixing element for fixing the bending sensor on the skin of the human body, wherein said fixing element is adapted to accommodate said bending sensor such that it is fixed only at one point of said fixing element and follows motions of that part of the human body on which said fixing element is fixed without following possible extensions of the skin of the human body.

### CROSS-REFERENCE TO RELATED APPLICATIONS

The present application is a continuation under 35 U.S.C. §120 of PCT Application No. PCT/EP2009/006806, filed Sep. 21, 2009, the entirety of which is hereby incorporated by reference.

### FIELD OF THE INVENTION

The present invention relates to a method and a system for detecting function parameters for the characterization of motion sequences at the human body, and to a computer-implemented method for the analysis of such function parameters.

### BACKGROUND OF THE INVENTION

Backache is a more or less strong ache of the human back which may have very different reasons. Physicians speak of dorsalgia or of lumbalgy if the loin-sacrum region is concerned. Probably the most frequent reason for a dorsalgia probably consists in a dysfunction of the joints in the region of the spine. However, approx. 90 percent of any chronical (recurrent or persistent) backache is still unspecific—i.e. in the scope of medical examinations no findings can ultimately be made which explain the disorders sufficiently. At present, it is not possible to objectively detect motion functions, for instance, for treating unspecific backache. Merely structural findings by means of imaging diagnostic methods are usual (which are, however, not relevant in the case of unspecific backache).

### SUMMARY OF THE INVENTION

It is therefore one of the objects of the present invention to provide a method and a system which enable the detection and, as the case may be, the analysis of function parameters for the characterization of motion sequences at the human body, so that unspecific or chronic disorders may be assigned to the motion patterns detected. The detection of the bending information is to take place predominantly in the region of the lumbar spine since the reason for a majority of any backache is to be seen here.

This and further objects are solved by providing a system for detecting function parameters for the characterization of motion sequences at the human body, comprising: a bending sensor with at least one strain gauge for detecting bending parameters, wherein said at least one strain gauge changes its impedance during an extension or compression, and sensor electronics for reading out and processing the bending parameters detected by said at least one strain gauge; and a fixing element for fixing the bending sensor on the skin of the human body, wherein said fixing element is adapted to accommodate said bending sensor such that it is fixed only at one point of said fixing element and follows motions of that part of the human body on which said fixing element is fixed without following possible extensions of the skin of the human body.

In another aspect, the invention can be a method for detecting function parameters for the characterization of motion sequences at the back of a carrier, comprising: providing a first and a second bending sensor each comprising at least one strain gauge and sensor electronics; providing a first and a second fixing element for fixing said first and said second bending sensors on the skin of the carrier; fixing said first and said second bending sensors on the back of the carrier by means of said first and said second fixing elements, wherein said first and said second bending sensors are arranged at the back in a V-shaped manner; detecting bending parameters by means of said at least one strain gauge of said first and said second bending sensors; and reading out and processing the bending parameters detected by said at least one strain gauge of said first and/or said second bending sensors by means of said sensor electronics of said first and/or said second bending sensors.

In a further aspect, the invention can be a computer-implemented method for the analysis of function parameters for the characterization of motion sequences at the human body, comprising: receiving of measurement values with bending parameters detected with a plurality of bending-sensitive segments of a first and a second bending sensor at different discrete points in time; converting the bending parameters to pertinent angles; forming angle sums for the first bending sensor for the different discrete points in time, wherein for each discrete point in time the sum is formed from the angles detected by the plurality of segments of the first bending sensor at the respective discrete point in time; forming of angle sums for the second bending sensor for the different discrete points in time, wherein for each discrete point in time the sum is formed from the angles detected by the plurality of segments of the second bending sensor at the respective discrete point in time; forming the difference from the angle sums of the first and the second bending sensors for the different discrete points in time, wherein for each discrete point in time the difference from the angle sum of the first bending sensor for the respective point in time and the angle sum of the second bending sensor for the respective point in time is formed; and generating a graphic illustration for at least one of the following data sets: the angle sums of the first bending sensor for the different discrete points in time; the angle sums of the second bending sensor for the different discrete points in time; the difference from the angle sums of the first and of the second bending sensors for the different discrete points in time.

### BRIEF DESCRIPTION OF THE DRAWINGS

Preferred embodiments of the instant invention will be described in the following with reference to the enclosed drawings.

FIG. 1 shows a system according to a preferred embodiment of the instant invention which comprises a memory unit 10 and two sensor bands 11a and 11b.

FIG. 2 shows a block diagram which schematically illustrates the electronic components of a preferred embodiment of the sensor band and their mutual connection.

FIG. 3 shows a block diagram which schematically illustrates the current supply of an embodiment of the sensor band 11.

FIGS. 4a and 4b show, in a strongly simplified form, the structure of an embodiment of the sensor band 11 in a lateral and a sectional view.

FIG. 4c shows the sensor electronics 41 of FIG. 4a in a sectional view.

FIG. 5 shows a sketch for the conversion of a curve into an angle.

FIG. 6 shows in a simplified illustration a preferred embodiment of a memory unit.

FIG. 7 shows a block diagram which schematically illustrates the electronic components of a preferred embodiment of the memory unit and their mutual connection.

FIG. 8 shows a block diagram which schematically illustrates the current supply of an embodiment of the memory unit.

FIGS. 9a and 9b schematically show a fixing plaster according to a preferred embodiment of the invention in a top view and a sectional view.

FIG. 10a shows a sketch which illustrates the overall structure of the fixing plaster according to FIG. 9 in a simplified manner.

FIG. 10b schematically shows the overall structure of the fixing plaster in a top view with the sensor band already being inserted into the plaster.

FIG. 11a and 11b show the patterns 102 and 103 of FIG. 10b, wherein the respective regions are indicated in which they are firmly connected to form a pattern 102, 103.

FIGS. 12a and 12b show the pattern 101 and the pattern 102, 103, wherein the respective regions are indicated in which they are firmly connected.

FIG. 13a shows a graph in which different angle sums measured with a parallel plaster arrangement are plotted over time.

FIG. 13b shows a graph in which different angle sums measured with a V-shaped plaster arrangement are plotted over time.

FIG. 14a schematically shows an arrangement of the memory unit 10 and the sensor bands 11a, 11b on the back of a carrier in accordance with a preferred embodiment of the invention.

FIG. 14b shows a rear view of a carrier on whose back reference points for the arranging of the sensor bands pursuant to FIG. 14a are marked.

FIG. 15 shows a sketch in which the position of a fixing plaster relative to the reference line 145 and the connection line 144a and 144b, respectively, of FIG. 14b is illustrated.

FIG. 16 shows a simplified flow chart for the interface between the embedded software and the user software.

FIG. 17 shows a simplified flow chart of the main program.

FIG. 18 shows a simplified flow chart of the interrupt service routine “Timer3-ISR”.

FIG. 19 shows a simplified flow chart of the interrupt service routines “I²C-ISR” and “UART-ISR.”

FIG. 20 shows an overview of various header and source code files of the embedded software of the memory unit.

FIG. 21 shows a simplified flow chart of the function “SD_Start.”

FIG. 22 shows a simplified flow chart of the function “memory.”

FIGS. 23a to 23d show a simplified flow chart of the function “main.”

FIG. 24 shows a simplified flow chart of the function “UART_Interrupt.”

FIG. 25 shows a simplified flow chart of the function “I²C-Interrupt.”

FIGS. 26a and 26b show a simplified flow chart of the function “Timer3-Interrupt”.

FIG. 27 shows an overview of various header and source code files of the embedded software of the sensor band.

FIG. 28 show a simplified flow chart of the function “main.”

FIG. 29 shows a simplified flow chart of the function “I²C-Interrupt.”

FIG. 30 shows an overview of the sub programs used in the live software.

FIG. 31 shows a first raw data plot in which angles are illustrated over time.

FIG. 32 shows a second raw data plot in which the current angles are illustrated segmentally.

FIG. 33 shows two plots in which the angle rate and the angle acceleration are each plotted over time.

FIG. 34 shows two dynamic plots in which the angle rate is plotted over the angle sum.

FIG. 35 shows a bar plot in which the current angles and accelerations of the position sensor are indicated.

FIG. 36 shows illustrations of the curvature areas for the right and the left sensors in which the current bending angles are visualized.

FIG. 37 shows an illustration in which the current angles and angle rates of the right and left sensors are shown and their maximum and minimum values are detected.

FIG. 38 shows a raw data illustration of the position sensor in which the results of the position sensors of both sensor bands are shown.

FIG. 39 shows an overview of the sub programs used in the analysis software.

FIG. 40 shows a sketch for explaining the terms direction of circulation, inflection point, and starting point which are used during envelope calculation.

FIG. 41 shows a raw data illustration in the analysis software in which the angle is plotted over time.

FIG. 42 shows dynamic plots in which the respective angle is plotted over the rate.

FIG. 43 shows rate illustrations in which the rate is plotted over time.

FIG. 44 shows graphs in which the measurement results of the position sensors are illustrated.

FIG. 45 shows a comparative illustration between the right and the left sensors in which the sensor segments of the right or left sensor, respectively, are each added and are illustrated with the sum of all sensor segments in a plot over time.

FIG. 46 shows level crossing graphs with and without activity, wherein the number of level crossings for the respective level are illustrated.

FIGS. 47a and 47b show intensity plots in two different possibilities of graphical illustration.

FIGS. 48a and 48b show two rate-time diagrams with an example of a “healthy” and an “ill motion.”

FIGS. 49a and 49b show two phase diagrams with an example of a “healthy” and an “ill motion.”

FIG. 50 shows an angle-time diagram of an exemplary motion in which a plurality of equidistant threshold levels for the angle are plotted.

FIG. 51 exemplarily shows a calculated relative arrangement of individual vertebrae in a schematic illustration.

### DETAILED DESCRIPTION OF THE INVENTION

The device or the system according to the present invention measures the function parameters for the characterization of motion sequences in the patient's everyday life. This has the advantage that he or she moves in a natural manner and in correspondence with his or her disease or pain situation during a measurement phase. For this reason, the following function features were attached particular importance to during the development of the device:
measurement is performed with high temporal and spatial resolution
long-time measurement up to 24 hours
live illustration
portable, small, slight (does not cause any restrictions as to mobility)
robust and thus suitable for daily use (work, sports, leisure time, sleep)
simple use and operation

The system according to the invention is to be considered as the “eye” of the therapist on the patient's back in an everyday situation. This system enables to register the motions of the patient in everyday life and to use them for an objective diagnostic assessment.

The system for detecting function parameters for the characterization of motion sequences at the human body in accordance with the invention may comprise: a bending sensor with at least one strain gauge for detecting bending parameters, wherein the at least one strain gauge changes its impedance during extension or compression, and with sensor electronics for reading out and processing the bending parameters detected by the at least one strain gauge; and a fixing element for fixing the bending sensor on the skin of the human body, wherein the fixing element is adapted to accommodate the bending sensor such that it is fixed only in one point of the fixing element and follows motions of the part of the human body on which the fixing element is fixed, without following possible extensions of the skin of the human body.

The system according to the invention is, for instance, intended to collect data regarding the bending of the back. Bending designates the deformation of a body under a load that acts vertically on the body axis. For detecting the dynamics of motion sequences at the human body (for instance, at the back in the region of the lumbar spine), one or a plurality of measuring sensors are to be fixed in an appropriate position on the skin by means of a fixing element, in particular a fixing plaster. The bending of the back results in an extension of the skin. Extension is the relative dimension change (prolongation or contraction) of a body under strain. If the dimension of the body increases, one speaks of a positive extension, otherwise of a negative extension or compression. The extension of the skin on bending of the back amounts to approx. 50% of length change in the region of the lumbar spine in the case of a flexion motion. Therefore, the fixing element is expediently flexible so that it does not loosen from the skin due to the length change discussed. The measuring sensors are fixed on the human skin (preferably at the left and at the right of the spine for detecting motions of the back) by means of the fixing elements. In so doing, the sensor band is connected with the fixing element via one point, also referred to as “reference point,” only and slides for the rest freely, preferably inside a cavity provided by the fixing element, so as to be able to follow the motions of the human body. In a preferred embodiment of the invention the cavity quasi forms a guide channel in which the sensor band is indeed held at the body to follow the bending thereof, but without having to follow the length change of the skin.

Strain gauges are measuring means for detecting extending deformations. They change their impedance already with slight deformations and are used as extension sensors. They thus detect a physical characteristic, an extension or a compression, and convert them into electric signals.

The electric impedance of the strain gauge changes with an extension or compression of the strain gauge. By measuring the impedance, in particular the electric resistance, the scope of the change of length of the strain gauge can be determined.

The bending sensor preferably comprises an elastically flexible substrate on which the strain gauges are fixed, e.g. glued. Once the strain gauge fixed on the substrate is, as is considered to be advantageous, disposed in a cavity forming a guide channel at the object to be examined, for instance, the back, a bending of the back will result in a corresponding bending of the substrate and thus in a change of length of the strain gauge.

The substrate used should therefore possess elastic properties. A work piece of an elastic material can be bent to a tension determined by the material (limit of elasticity) to then return elastically to the initial state without permanent deformation. As a substrate material, spring band steel or FR 4 is, for instance, suitable. FR 4 is a designation for the fire resistance of the conductor board material; FR 4 is the common standard for consumer electronics.

In accordance with a preferred embodiment of the instant invention, the bending sensor comprises a plurality of strain gauges, wherein every two strain gauges are fixed on opposite sides of the substrate such that both strain gauges substantially detect the same bending of the substrate which follows the bending of the object of examination, for instance, the back.

Since the bending of the substrate is measured simultaneously by two strain gauges on opposite sides of the substrate, it is possible to effectively compensate for disturbance variables by means of a bridge circuit, e.g. a Wheatstone measuring bridge, which forms a difference signal from the signals of the two strain gauges, and to amplify the actual measuring signal. Failure influences such as tensile or compressive strengths as well as temperature fluctuations which may possibly cause additional changes of length of the substrate are compensated for since they influence the measurement results of both strain gauges in equal measure and are thus compensated for when the difference from the two measuring signals is formed. The actual measuring signal is, however, amplified since a bending of the substrate causes a positive change of length of the one strain gauge and a negative change of length of the other strain gauge, wherein both changes of length have substantially the same value. During the calculation of the difference the measuring signal is thus substantially doubled. It is consequently possible to determine the actual bending with higher accuracy and reliability.

In a preferred embodiment the bending sensor comprises a plurality of strain gauges for detecting the bending parameters in respectively different measuring zones. The strain gauges may be arranged in a cascaded or an overlapping manner. It is thus possible to metrologically detect the bending information resulting from the body motion irrespective of the location.

Every bending-sensitive measuring zone detects the respective curvature applied in its measuring section in a positive or negative bending direction in at least one spatial plane. The strain gauges arranged in the different measuring zones are preferably triggered in a temporally offset manner, so that the hardware effort and the current consumption are considerably reduced. In particular only one analog-digital converter and only one return line are needed for all strain gauges of a substrate side since only data of one single measuring sensor are read out at each point in time. Simultaneously, the read-out frequency is large enough to detect the dynamics of the motion irrespective of the location. For instance, with a trigger frequency of 1 kHz and, for instance, 10 bending zones, a read-out frequency of 100 Hz becomes possible, which enables a dynamic detection of motion (rate, acceleration of bending).

Furthermore, the system according to the invention may advantageously comprise a position sensor for detecting the position of the sensor band relative to the gravitation field of the earth or to the earth's magnetic field. The gravitation field of the earth predetermines a constant direction. The measurement of the bending irrespective of the location enables to detect the bending in a particular plane relative to the direction of the gravitation field. Thus, it is, for instance, possible to determine how the back is bent for the lifting of loads.

The position sensor may also predetermine a starting vector for the detection of the bending information. The position sensor detects the position of the sensor at the back prior to the beginning of the bending measurement. The subsequently detected bending constitutes a relative deviation vis-à-vis the orientation of the starting vector.

The data detected by the detector are preferably output as digitized electronic signals by the sensor electronics and are stored advantageously in a memory unit in an electronic data memory. As a data memory, a flash memory such as a SD memory card or a micro SD card may, for instance, be used. The flash memory offers the advantage that it is relatively small and light and preserves the stored data without the data memory having to be connected to a power supply.

The bending parameters detected are preferably used to determine a plurality of dynamic parameters. In particular the bending angle as a function of the time and/or the place, the bending rate as a function of the time and/or the place, the bending acceleration as a function of the time and/or the place, the Fourier transformation of the functions of the bending angle, the bending rate, and/or the bending acceleration can be derived.

The detecting of bending parameters of the bending sensor is preferably performed over a period of at least 24 hours to enable a long-time analysis. The application may consequently be performed in analogy to the long-time ECG measurements at arbitrary periods up to 24 hours. The detecting of bending parameters may in particular be performed in addition to treatment so as to detect a positive or a negative correlation between therapeutic measures and the motion parameters detected.

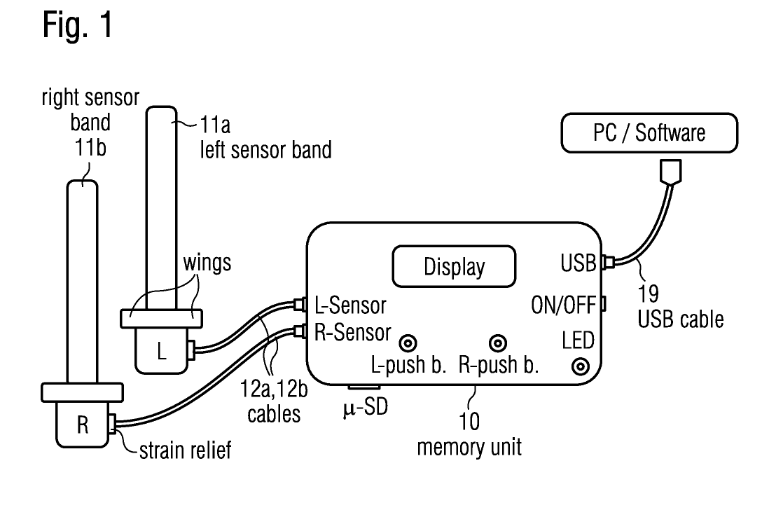

FIG. 1 shows a system according to a preferred embodiment of the instant invention which comprises a memory unit 10 and two sensor bands 11a and 11b which are each connected via a cable 12a, 12b, e.g. a 4-pole cable, with the memory unit 10 for current supply and data transmission. Advantageously, the system may comprise further accessories such as, for instance:
a USB data cable 19
a USB Bluetooth module
a case
fixing plasters
a memory card (e.g. a 1 GB, 2 GB, or 4 GB micro-SD memory card)
user software

The two sensor bands 11a, 11b are constructed with substantially identical hardware components. The differences between the two sensors lie in the embedded software and a solder bridge in the sensor electronics. In the embedded software the address for sensor differentiation (left/right) is stored, and with the solder bridge the address of the EEPROM is defined.

The sensor electronics convert the motion of the back by means of strain gauges to electrically detectable values which are amplified and digitized. Advantageously, an integrated position sensor detects the position of the sensor band 11 with respect to the earth gravitation. All the measured data can be transferred to the memory unit 10 via the cable connection 12.

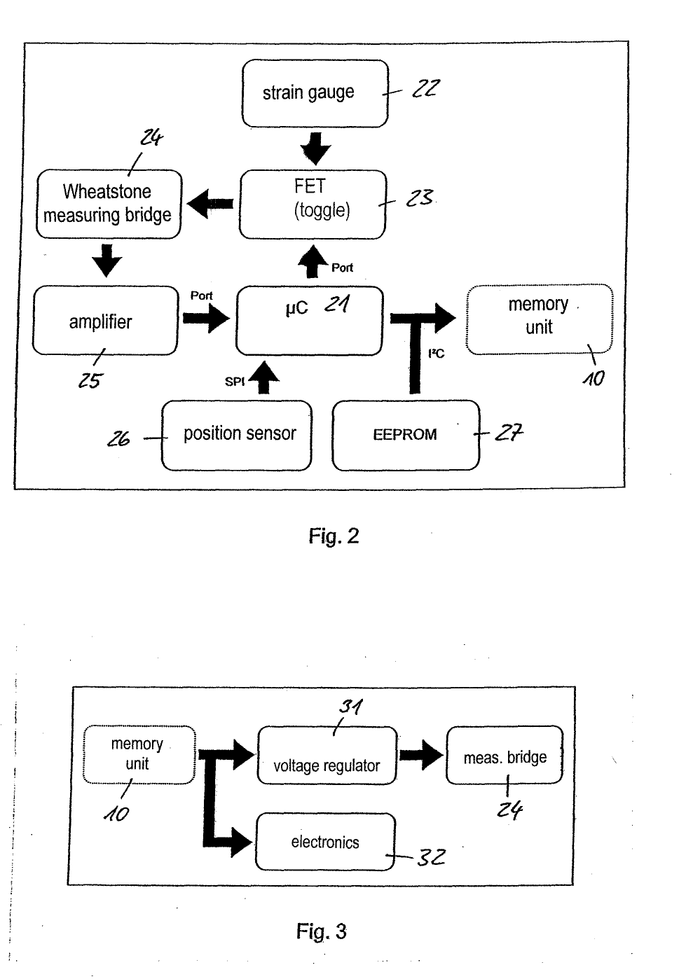

FIG. 2 is a block diagram that schematically illustrates the electronic components of a preferred embodiment of the sensor band and their mutual connection.

In the sensor band 11 a microcontroller (μC) is used whose object is the detection of measurement values and the transmission of measured data to the memory unit 10. On instruction of the memory unit 10 the program flow is initiated. Some of the objects of the μC are:
triggering the measuring bridges of the strain gauges;
reading in and digitizing the measured data of the strain gauges;
reading in the measured data of the position sensor;
storing the detected measured data; and
sending the detected measured data to the memory unit.

The digitizing of the measured data is, for instance, performed with an internal 12 bit A/D converter of the μC. An external reference component supplies a reference voltage of, for instance, 2.5 V.

The strain gauges 22 convert the bending of the sensor band to an electrically detectable magnitude. In so doing, the change of resistance as a function of the extension of the strain gauge 22 is used.

By means of the FETs, the strain gauges are connected to the measuring bridge. This is performed sequentially with every two opposite strain gauges. Thus, a plurality of measuring bridges are obtained, only one of which is active at a time.

By the use of two strain gauges in a semi measuring bridge 24, disturbance variables such as temperature and electromagnetic radiation are largely compensated for. Additionally, a stronger measuring signal is obtained since it is additively composed of both strain gauges.

A differential amplifier 25 is used as an amplifier. It amplifies the bridge voltage and generates an output voltage relating to the mass which is adapted to be processed by the A/D converter.

The position sensor 26 used renders information about the position with respect to the earth's gravitation. On request, measurement values for the acceleration in X-, Y-, and Z-direction can be read out digitally. The sensor is triggered and read out by the μC via a SPI interface. The acceleration is preferably measurable up to maximally the eightfold gravitational acceleration (G) with a resolution of, for instance 8 bit.

The EEPROM memory 27 serves as a storage of the calibration data. It is, for instance, controlled and read out via an I²C (Inter-Integrated Circuit) interface by the μC of the memory unit. Additionally, an identification number and a mark for the respective sensor, e.g. “L” for left sensor or “R” for right sensor, may, for instance, be stored in it. Since two sensor bands are positioned at an I²C interface, it has to be ensured that the μC of the memory unit is capable of differentiating them. This is, for instance, implemented by different defining of the EEPROM addresses by means of solder bridges on the electronic conductor board.

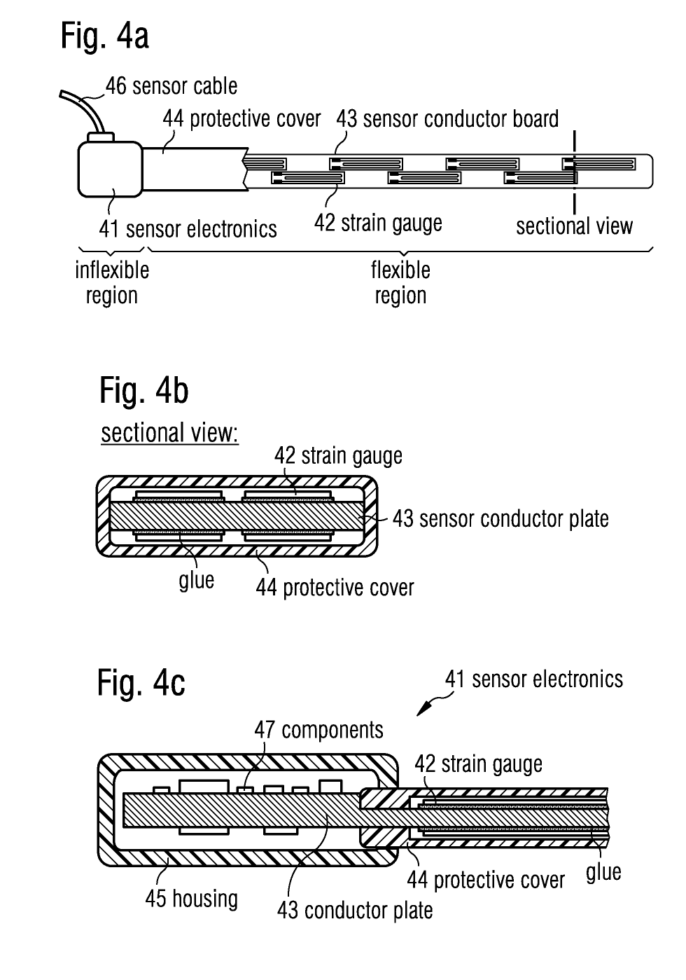

FIG. 3 is a block diagram that schematically illustrates the current supply of an embodiment of the sensor band 11.

A voltage regulator 31 is used as a voltage source for the measuring bridge. It supplies a constant voltage, e.g. 3.0 V, with which interferences of the measuring signal are largely minimized.

The remaining electronics 32 on the sensor band, such as μC 21, EEPROM 27, amplifier 25, reference voltage source, and position sensor 26, are supplied directly by the voltage regulator of the memory unit with a voltage of, for instance, 3.3 V.

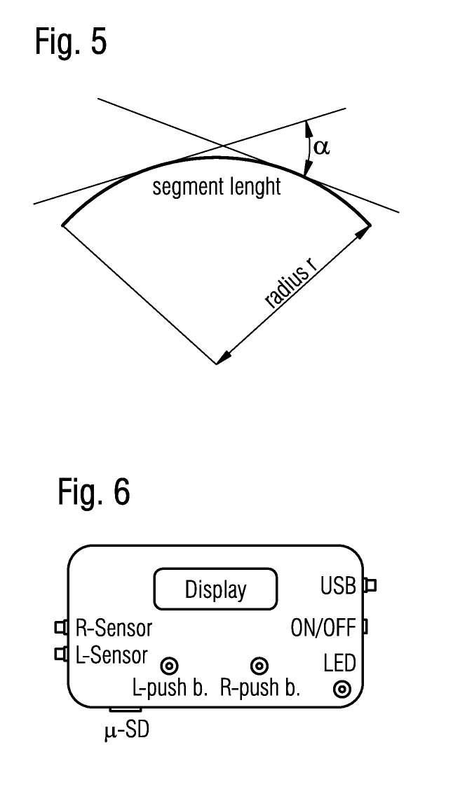

FIGS. 4a and 4b illustrate in a strongly simplified form the structure of an embodiment of the sensor band 11 in a lateral and a sectional view. As a basic material for the sensor band 11, an electronic conductor board 43 is preferably used. It consists of a substantially inflexible, e.g. approx. 1.5 mm thick region on which the sensor electronics 41 is fitted, and a flexible, e.g. approx. 0.5 mm thick, bending-sensitive region on which the strain gauges are glued.

The strain gauges 42 are each applied at the top and at the bottom congruently in pairs. Every strain gauge 42 preferably has a length of, for instance, 50 mm. On each side of the substrate, preferably 7 strain gauges are applied without gap, so that a bending-sensitive length of 350 mm is generated in the instant example. It is, however, also conceivable to use a different number of strain gauges, e.g. 3, 4, 5, 6, 8, 9 or more strain gauges, wherein also the length of the strain gauges is not predetermined to the preferred length of 50 mm, but wherein the strain gauges may also be shorter or longer than 50 mm.

The electric contact between the strain gauges 42 and the conductor paths on the sensor conductor board 43 may, for instance, be established by the soldering of a copper foil strip having e.g. a strength of approx. 50 μm. The inflexible region of the conductor board 43 with the sensor electronics 41 is located in the bottom portion of the sensor band. The conductor board 43 is preferably fitted with electronic components 47 on both sides. The electric connection with the memory unit 10 is preferably established with a 4-pole cable 46 which is used both for data transmission and for current supply for the sensor band.

FIG. 4c shows the sensor electronics 41 of FIG. 4a in a sectional view. The sensor electronics 41 is protected from mechanical, chemical, and electrical environmental influences by means of a housing 45. The housing 45 consists advantageously of two shells that are connected with or screwed to one another. The cable 46 at the sensor band is conducted outside via an outlet at the housing shells. For the cable 46 a strain relief is preferably provided, wherein the outlet at the housing 45 may, for instance, serve as a strain relief (see FIG. 1), wherein a suitable ring or an aluminum ring, respectively, is drawn over the outlet and presses the two housing parts against the cable. Additionally two wings (see FIG. 1) are applied at the sensor housing so as to be able to fix the sensor to the fixing plaster.

The protective cover 44 preferably consists of a so-called “heat shrinkable tubing”, i.e. a plastic tube that contracts strongly under the influence of heat. It protects the strain gauge 42 from mechanical and chemical environmental influences and cares for the necessary sliding ability in the fixing plaster due to its smooth surface.

Due to the manufacturing, every sensor band has tolerances that have an effect on the measurement values. Consequently, different measurement results are obtained with different sensor bands at equal measurement conditions. To make the sensor bands metrologically comparable with each other and to be able to map reality, they are calibrated.

The sensor band reproducibly converts bending information to a voltage value. This is then converted to the desired measurand, in the instant case an angle. A caliber may, for instance, serve this purpose.

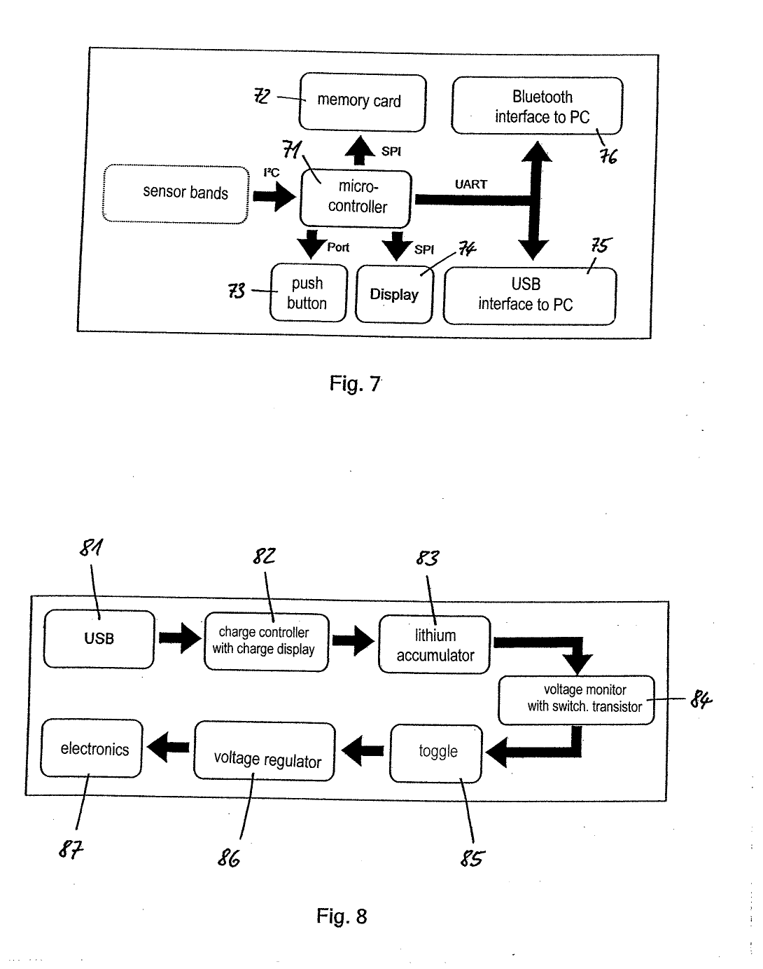

The bending assigned to an angle may be determined from the segment length and the radius (see FIG. 5) pursuant to the following formula:

$$angle = \frac{length of segment \cdot 360 °}{2 \cdot π \cdot radius}$$

For calibration, the corresponding measurement value is taken and stored from each segment of the sensor band preferably for three angles (e.g. 12°, 0°, and −12°). The respective angles may advantageously be adjusted by means of a caliber comprising, for instance, several slits (e.g. 0° (straight line), −12°/12° with a radius of e.g. 238.7 mm) with different, suitable curves. The negative bending direction may be implemented in that the sensor band is reversely pushed into the slit. The measured data obtained are stored in the sensor band on the EEPROM. From these, the user program calculates two interpolation straight lines (in the instant example from −12° to 0° and from 0° to 12°) by means of which it is possible to convert any bending to the corresponding angle.

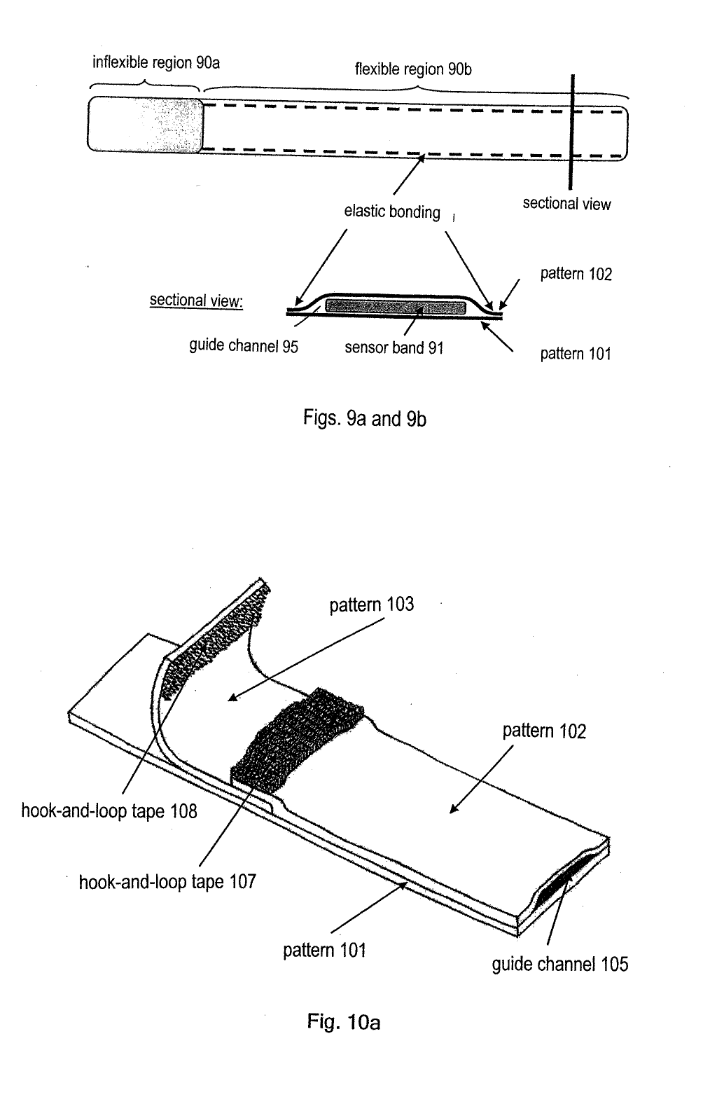

The memory unit comprises a voltage supply (e.g. an accumulator) and controls the measuring sequences. FIG. 6 shows in a simplified illustration a preferred embodiment of a memory unit with the display and operation elements thereof.

The memory unit controls both sensor bands and advantageously transmits the measured data to a user computer. Additionally, the measured data may be stored on an exchangeable memory card. To measure the bending motion and the space orientation with high definition, the measured data are advantageously detected with a sampling rate of e.g. 100 Hz. Via two push-buttons and one display the device is operated or status information is obtained, respectively.

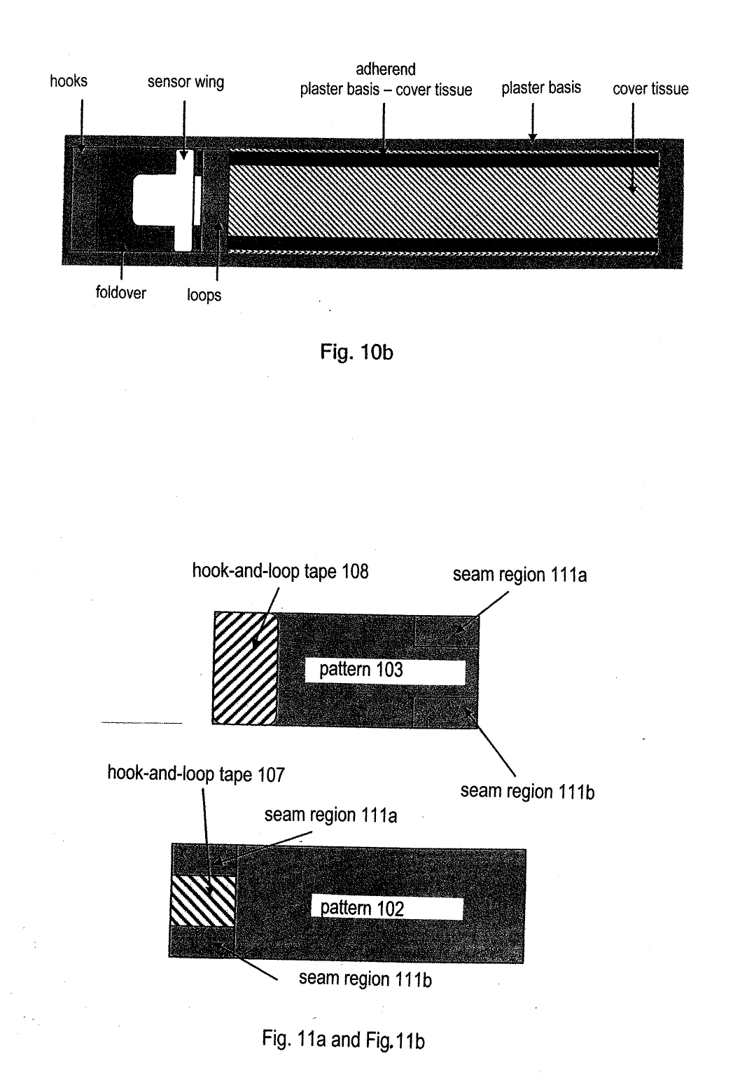

FIG. 7 is a block diagram schematically illustrating the electronic components of a preferred embodiment of the memory unit and their mutual connection.

In a particularly preferred embodiment, the C8051F410 by “Silicon Laboratories” is, for instance, used as a μC. It comprises several interfaces such as I²C, SPI, and UART which serve for communication with the periphery (see FIG. 7). It is clocked by an external quartz, so that a high-precision time basis is generated. Due to the characteristics mentioned, this μC is particularly well suited. However, any other μCs may, of course, also be used which possess the performance features required for this application. The μC in the memory unit controls the operation and display elements, the storage of the digital measured data, the communication with the user software, and the communication with the two sensor bands.

A μSD card (1 GB, 2 GB, or 4 GB) 72 ensuring a measurement duration of at least 24 hours serves, for instance, as a memory for the measured data taken. Via the SPI interface the μC controls the memory card on which the measured data are stored.

The two push-buttons 73 serve for input purposes. They are directly connected to two ports of the μC. On actuation they change the level from high to low.

The, for instance, alphanumerical display that is addressed via the SPI interface serves as a display element 74. It is equipped with a background illumination that is adapted to be activated by the μC via a transistor.

The data transmission to the user software may optionally take place via Bluetooth 75 or USB (Universal Serial Bus) 76. Both interfaces are controlled by the microcontroller via UART (Universal Asynchronous Receiver/Transmitter). In so doing, the data traffic may be switched via a multiplexer to one of the two interfaces. The USB interface has priority, this means as soon as the memory unit is connected to a PC via USB, it is active and Bluetooth is deactivated. To save current, the Bluetooth module is deactivated by a transistor in the case of inactivity, and the UART/USB interface converter is supplied via the connected PC.

The memory unit is connected with two sensor bands via the I²C interface.

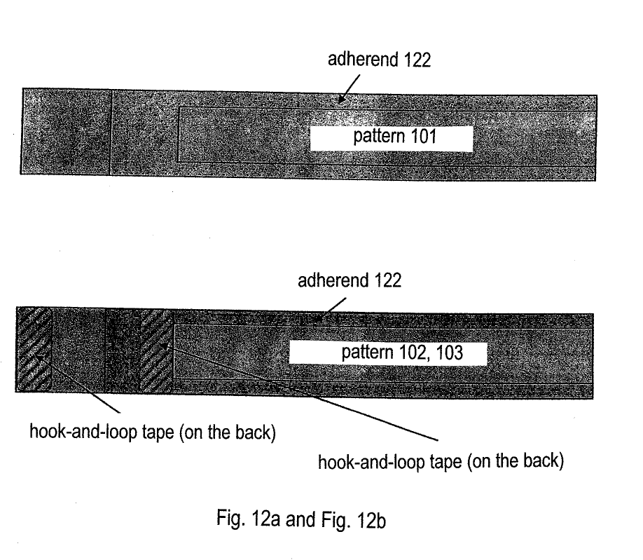

FIG. 8 is a block diagram schematically illustrating the current supply of an embodiment of the memory unit. The accumulator may advantageously be charged via a mini USB socket 81 at the memory unit. Charging may be effected, for instance, via a USB charge adapter or directly at the PC.

The charge controller 82 independently controls the charge in the case of a plugged USB cable. An incorporated LED (light emitting diode) advantageously symbolizes the charge.

The complete system is supplied by a suitable accumulator, e.g. a 3.7 V lithium polymer accumulator 83 with a capacitance of at least 1500 mAh. The accumulator should be adapted to enable an operation of the system of at least 24 hours.

The state of the accumulator is advantageously displayed by a percent indication at the display. For calculation of the capacitance of the accumulator the accumulator voltage may be measured by a voltage divider.

The voltage monitor with switching transistor 84 has the object to protect the accumulator from total discharge. If the lower voltage limit of, for instance, approx. 3.3 V is achieved at the accumulator, the voltage monitor disconnects the entire electronics from the accumulator via the switching transistor. This protects the accumulator from irreparable damage. The upper voltage limit at which the electronics is again supplied with current lies, for instance, at approx. 4 V.

The toggle 85 serves for the manual switching on or off of the system. On switching off it disconnects the accumulator voltage from the electronics.

The voltage regulator 86 converts the accumulator voltage to a constant voltage, e.g. 3.3 V. This voltage is used as a supply voltage for the entire system including the sensor bands.

The user obtains status information via the display and may operate the device, for instance, via two push-buttons and one toggle. The following functions may, for instance, be chosen:
Switch on and off memory unit with I/O toggle
Activate display illumination via push-button operation (automatic switch-off after 3.5 sec.)
Switch on/off Bluetooth interface with left push-button
Set pain point with right push-button

The capacitance of the accumulator determines the operating time of the system. It is calculated pursuant to the following formula:

accumulator capacitance=current*operating time

With an operating time of at least 24 hours and an average power requirement of approx. 26 mA in the mode “Bluetooth off” and data storage on the memory card, an accumulator capacitance of approx. 624 mAh results. In the mode “Bluetooth on” and data storage on the memory card, the power requirement is approx. 50 mA, which requires an accumulator capacitance of 1200 mA with equal operating time. An accumulator with a capacitance of at least 1500 mAh is, for instance, used.

The capacity of the memory card should be dimensioned such that data can be stored thereon for 24 hours at least. The formula for the calculation reads:

storage capacity=data set volume per second*3600*24

With a data set volume of e.g. 41 bytes per data set in 10 ms (for e.g. 100 Hz) there results a data amount of 354.24 MB after 24 hours. A memory card with a storage capacity of at least 1 GB is, for instance, used.

The memory unit may advantageously be carried in a bag, for instance, of synthetic leather, at the left hip. The bag is provided with a suitable fastening means, so that it is adapted to be fastened to a belt. By suitable means, e.g. hook-and-loop fasteners, the bag may be opened and closed.

The fixing plaster establishes the connection of the sensor bands with the human back. It couples the sensor band with the mechanical system “carrier's back”, i.e. the back of the person carrying the system according to the invention. The sensor band comprises an elastic guide channel in which the flexible region of the sensor band may slide during a flexion or extension motion of the back. With the fixing plaster the sensor band (especially the sensor electronics) is fixed on the back and is closely pressed to the skin surface with every bending position of the back.

In the following, a particularly preferred embodiment for the fixing plaster will be described. It is noted that the fixing plaster described in the following merely constitutes one embodiment out of a plurality of possible embodiments for a fixing plaster according to the invention. Thus, this embodiment has exemplary character only, and the scope of protection of the instant invention is not restricted by the optional features indicated in the following.

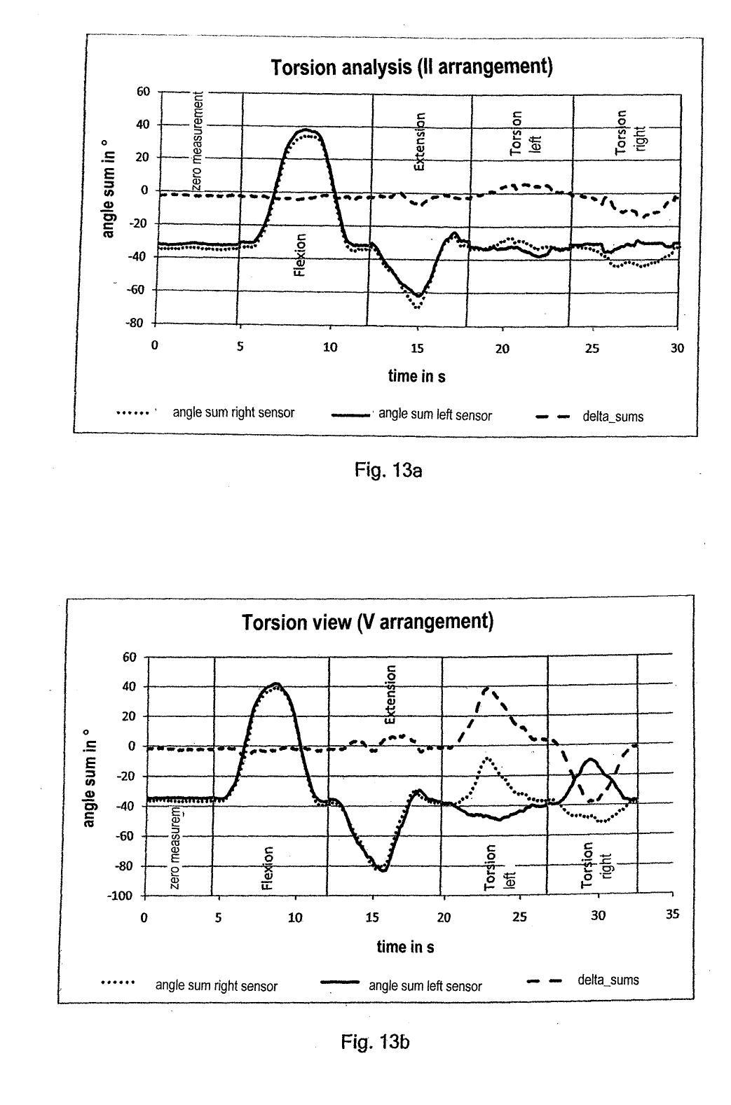

FIGS. 9a and 9b schematically show a fixing plaster according to a preferred embodiment of the invention in a top view and a sectional view. The fixing plaster advantageously comprises a flexible region 90b and an inflexible region 90a.

The inflexible region fixes the sensor electronics to the fixing plaster to ensure the local position of the sensor electronics with respect to two bottom reference points P_{BL} and P_{BR}.

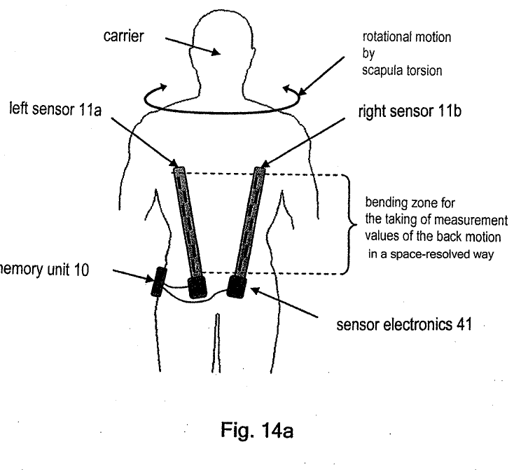

The flexible region of the fixing plaster is provided with a guide channel 95 in which the sensor band 91 may slide. The guide channel may, for instance, be implemented by two fabric layers (see FIG. 10) that are adapted to be extended in longitudinal direction and that are glued with each other. This ensures that the fixing plaster extends or compresses, respectively, along with the skin surface in any position of the back and adapts itself ideally to the shape of the back.

FIG. 10a shows a sketch that illustrates the total structure of the fixing plaster of FIG. 9 in a simplified manner. The fixing plaster comprises three main components referred to as (first) pattern 101, (second) pattern 102 and (third) pattern 103 in the following. FIG. 10b schematically shows the total structure of the fixing plaster of FIG. 9 in a top view, with the sensor band already being inserted.

The fixing plaster is adhered to the human back. It is therefore necessary that at least the materials used in pattern 101 are biocompatible. The plaster is designed elastically, so that it is capable of imitating changes of length of the skin during motion. It is advantageously designed such that the plaster loses its adhesiveness after the first detaching from the skin, so that it is prevented from being used a second time. Advantageously, the plaster is further designed such that the full adhesiveness is achieved some minutes after the application and lasts for such a long time that the plaster reliably adheres to the human skin for at least 24 hours.

The fixing plaster fixes the sensor band in the bottom region of the back. It has the object of locally retaining the sensor band with the plaster component “pattern 103”. This is effected in that the sensor part positioned there is strongly enclosed by the material. The two hook-and-loop tapes 107 and 108 establish a strong, but detachable connection.

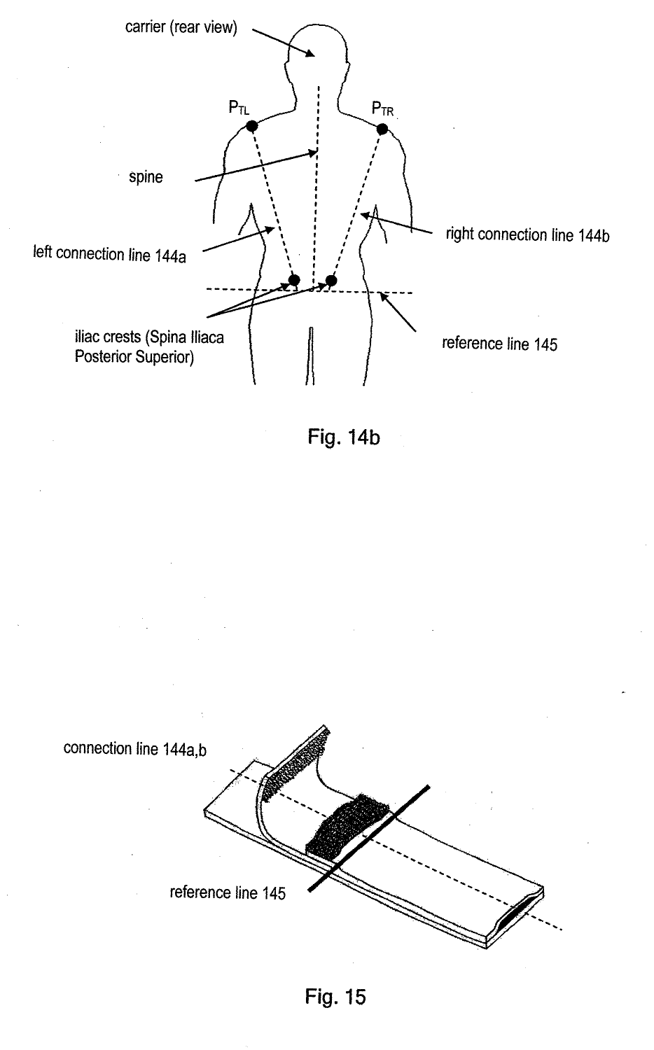

The seam regions 111a and 111b (see FIGS. 11a and 11b) are designed tearproof and serve as a stop for the sensor portion enclosed by the pattern 103. For this purpose, the sensor housing comprises small lateral projections, so-called “wings” (see FIG. 10b). The sensor is insertable into the guide channel 105 up to the stop. By means of the hook-and-loop fasteners 107 and 108 a bag is formed which prevents the sensor from slipping out. The guide channel 105 is formed by the materials of patterns 101 and 102. The sensor portion to be inserted into the guide channel 105 is to be retained closely to the human skin with every position of the back. This requirement applies for the entire length of the guide channel 105.

The guide channel 105 is to be dimensioned such that the sensor band is able to slide therein and is designed such that it neither constricts nor widens (positively and negatively) by a change in length.

The carrier does not feel the fixing plaster or will forget it after a short time of getting accustomed (e.g. <30 min), respectively.

The pattern 101 is provided with an adherend on one side thereof. The adherend extends substantially over the entire surface of the material and is protected with a foil for stripping off. The material of pattern 101 is biocompatible. It is elastic in longitudinal direction only. It imitates the extension or compression of the skin in the case of an extension or flexion motion of the human back.

The material of pattern 102 is advantageously also biocompatible. It is elastic in longitudinal direction and follows the extension or compression of the skin in the case of an extension or flexion motion of the human back. It retains the sensor close to the pattern 101 and thus to the skin. The pattern 102 comprises a hook-and-loop tape 107 as to be seen in FIG. 11b. The pattern 102 consists preferably of monoelastic jersey and is sewn onto pattern 103. Sewing is performed at the regions 111a and 111b (see FIG. 11a).

The material of pattern 103 is advantageously also biocompatible. It is inelastic in the longitudinal and transverse directions and comprises a hook-and-loop tape 108a, as to be seen in FIG. 11a. The pattern 103 is sewn underneath pattern 102. Sewing is performed at the regions 111a and 111b (see FIG. 11b). The pattern is, for instance, manufactured of jeans fabric with a 100 percent cotton share.

Pattern 102 is sewn on pattern 103. The seam regions 111a and 111b of patterns 102 and 103 have to be placed upon each other congruently during sewing. After the sewing of pattern 102 on pattern 103 a plaster component is obtained with is referred to as pattern 102, 103 in the following. Both hook-and-loop tapes 107 and 108 are positioned on the same surface side of pattern 102, 103 after sewing.

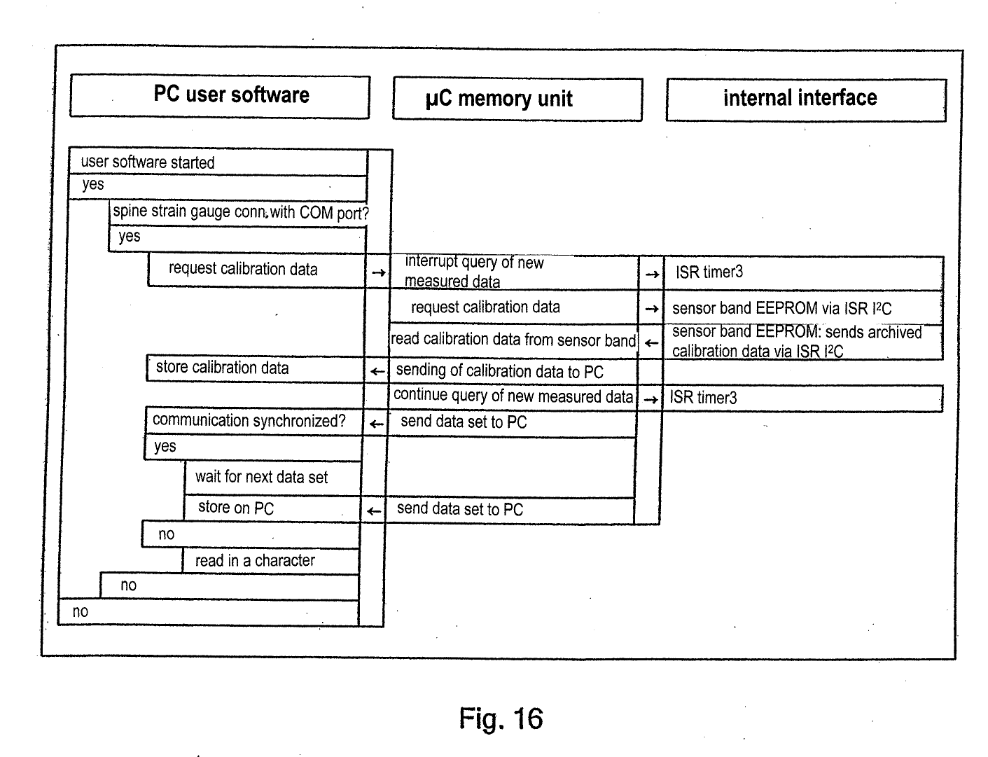

The pattern 102, 103 is glued on pattern 101 pursuant to the illustrations in FIGS. 12a and 12b. The adherend 122 of the patterns 101 and 102, 103 is placed upon each other congruently during gluing. A glue is used which does not restrict the elasticity of the materials glued with one another.

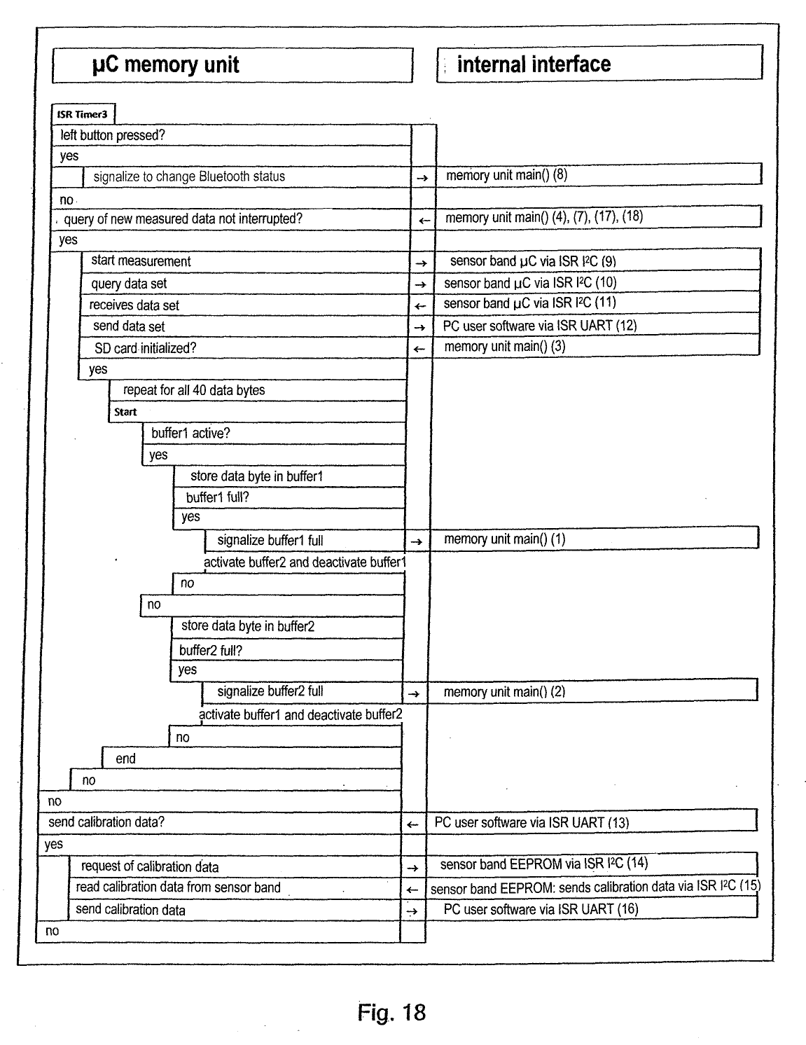

FIG. 14a schematically illustrates the arrangement of the memory unit 10 and of the sensor bands 11a, 11b, each with the sensor electronics 41. In accordance with the invention, the two sensor bands 11a, 11b are fastened in a V-shaped manner at the human back, and the memory unit 10 is preferably applied at the left hip. For applying the sensor bands on the human back, fixing plasters, for instance, the above-described preferred embodiments for fixing plasters, are used.

The system will no longer be noticed by the carrier after a time of getting accustomed of <30 minutes and influences the human musculosceletal system neither actively nor passively. By the comparison of the bending information between the right and the left sensor bands it is possible to draw conclusions on torsion motions of the body.

In accordance with the invention, the fixing plasters are adhered to the skin of the back of the carrier at the left and at the right next to the spine in a V-shaped manner. The V-shaped arrangement of the sensors has proved to be particularly advantageous since torsions (i.e. rotational motions of the shoulder) may also be detected with this arrangement.

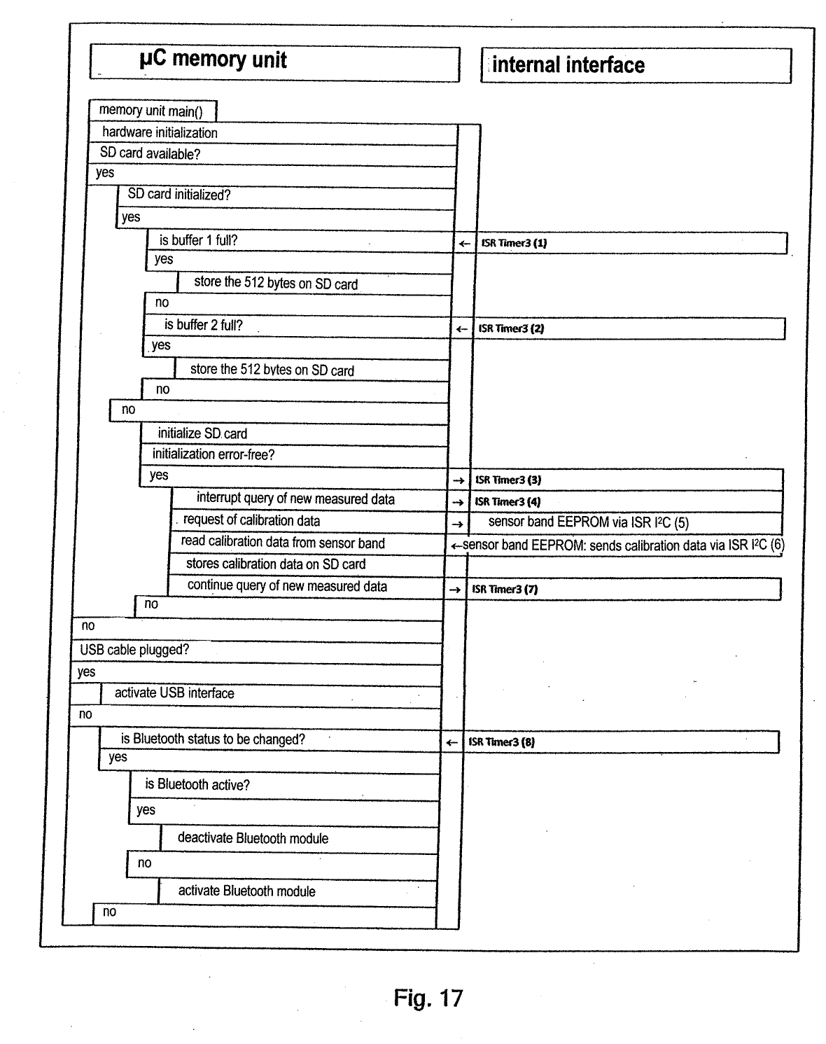

FIG. 13a shows a graph in which the angle sum is plotted over time. A zero measurement, a flexion, an extension, a torsion to the left, and a torsion to the right were successively measured with a parallel plaster arrangement. Both the angle sum of the right sensor and of the left sensor is plotted individually each, and the difference of the angle sums of the two sensors. It becomes evident that, with this arrangement, it is possible to detect flexion and extension motions well, but torsion motions only very poorly. The difference value of the angle sums shows only slight amplitudes for the torsion motions, too.

FIG. 13b shows a graph in which, in analogy to FIG. 13a, both the angle sum of the right sensor and of the left sensor is plotted individually each, and the difference of the angle sums of the two sensors. Here, too, a zero measurement, a flexion, an extension, a torsion to the left, and a torsion to the right were measured successively, but with a V-shaped sensor arrangement. In this graph, both the flexion and extension motions and the torsion motions can be recognized well. In particular in the plot of the difference of the angle sums of the two sensors the peaks for the torsion motions are distinctly pronounced and can thus be detected well. Test measurements have revealed that the peaks of the difference value of the angle sums of the two sensors were pronounced up to 4 times stronger in the case of a V-shaped arrangement of the sensors than in the case of a parallel sensor arrangement.

For the application of the plasters it is of advantage to orientate the plaster with respect to reference points at the carrier which are easy to touch, so as to ensure the same conditions, in this case substantially the same arrangement, with every application. In the following, a particularly advantageous embodiment of the present invention is described which meets the above-mentioned requirements. Other embodiments in which, for instance, the plasters are integrated in a model by means of which the position of the sensors or plasters on the back is easy to reproduce are, however, also conceivable.

FIG. 14b shows a rear view of the carrier on which reference points are marked which have turned out to be particularly advantageous for the application of the fixing plasters. As top reference points P_{TL} (left top reference point) and P_{TR} (right top reference point) serve the two touchable outer ends of the spina capulae (akromion) with a normal position of the scapulae. The reference line is positioned 5 cm below the touchable iliac crests 141a and 141b (spina iliaca posterior superior).

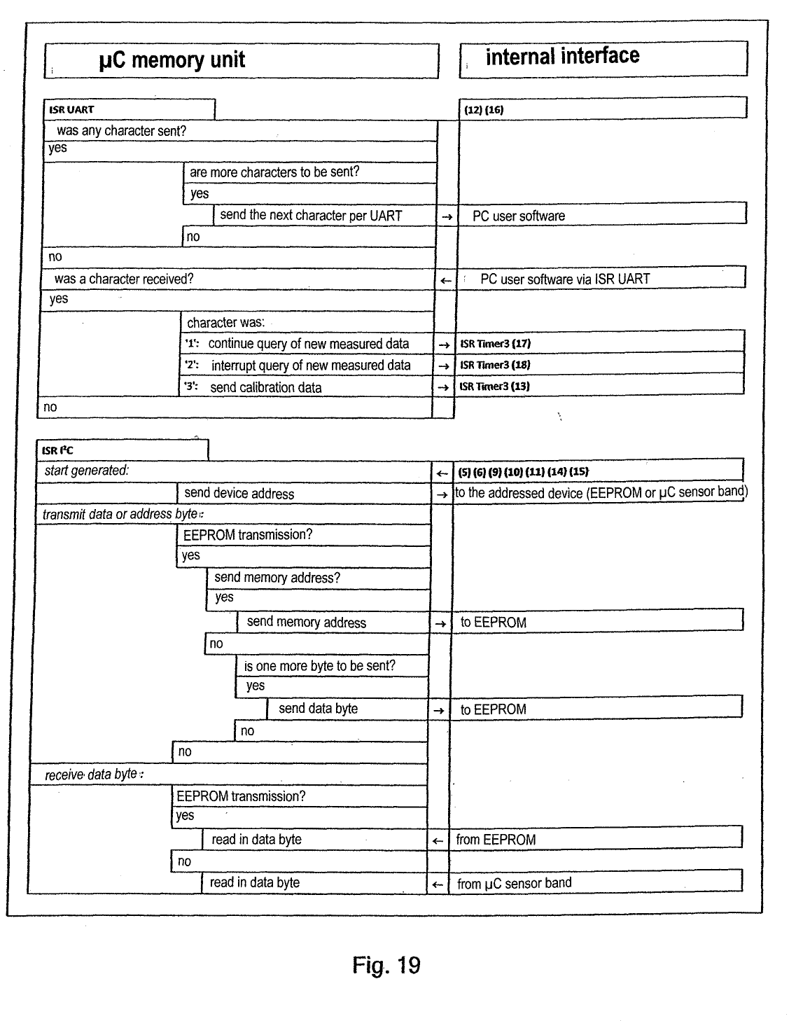

The position of the fixing plasters relative to the reference line 145 and the connection line 144a or 114b, respectively, is illustrated in the sketch shown in FIG. 15. The symmetry axis of the fixing plaster lies on the connection line 144a or 144b, respectively, while the reference line crosses the symmetry axis of the plaster in the vicinity of the hook-and-loop fastener. The plasters are adhered in a mirror-symmetrical manner to the spine along the connection lines.

When adhering the fixing plasters to the skin of the carrier care has to be taken that the plaster is not or just slightly stretched. In the case of strong hair-growth it is expedient that the carrier is shaved in the appropriate regions. For optimum hold, the skin surface should not be sweaty or oily during the plaster application. The adhesion is preferably performed from the bottom to the top. Once the plaster is applied completely, it is shortly massaged in. During this time the carrier advantageously performs some flexion and extension motions.

After the adhesion of the fixing plasters the sensor bands may be applied. It is expedient to introduce the sensor bands only after a waiting time of at least 15 minutes into the fixing plasters since the adhesiveness of the plasters will have developed sufficiently by then.

To facilitate the introducing of the sensor bands, the carrier holds the upper part of his or her body slightly bent forward. The sensor is introduced into the guide channel of the fixing plaster up to the stop. In so doing, care has to be taken that the left and the right sensors are not confused. For this purpose it is expedient that every sensor band is appropriately marked on the housing, e.g. by “left” or “right”. Furthermore, care has to be taken that the sensor cables exit at the left side (see FIG. 14a).

The cables of the sensor bands are connected with the connectors at the memory unit. Expediently, a suitable marking of the connectors for the left or the right sensor, respectively, is available on the housing of the memory unit.

The subject advantageously carries the memory unit in a belt bag at his or her left hip side. The connector sockets of the memory unit point from the carrier's view backwards. Thus it is possible to connect the cables of the sensors to the memory unit without any loop formation.

In the memory unit an embedded software controls the reading out and the storing of the digitized measured data, the functionality of the operation and display elements, and the transfer of the measured data. Time-critical processes such as the starting of a new AD-conversion (e.g. every 10 ms), the reading out of measured data from the sensors, or the sending of the measured data to a PC, are treated in a preferred manner. Also the priorities of the time-critical processes are hierarchized among each other. In the sensor band, the embedded software controls the reading out, converting, and digitizing of the analog measured data of the sensor bands.

In the following, a preferred embodiment of the software control will be described in detail. This embodiment only serves for explanation and has a purely exemplary character. It is noted that the scope of protection of the present invention is not to be defined or restricted on the basis of the features of this embodiment.

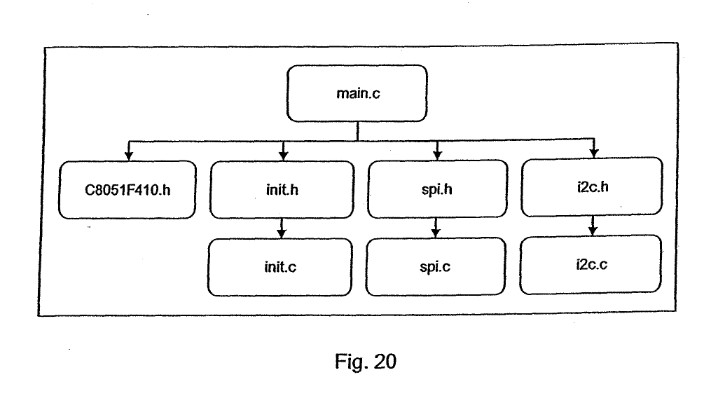

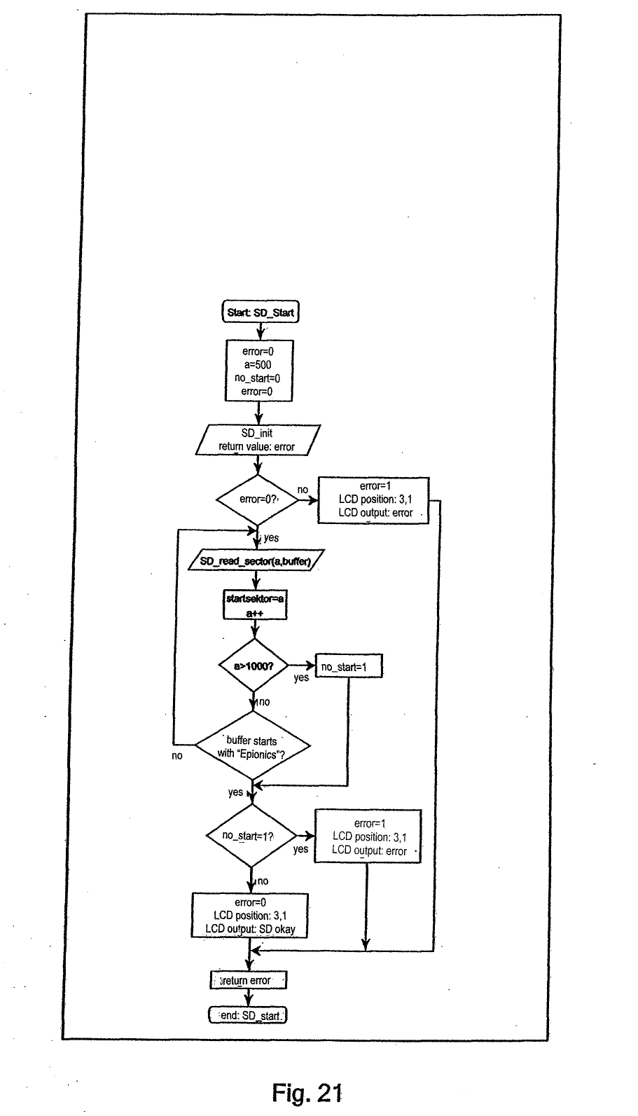

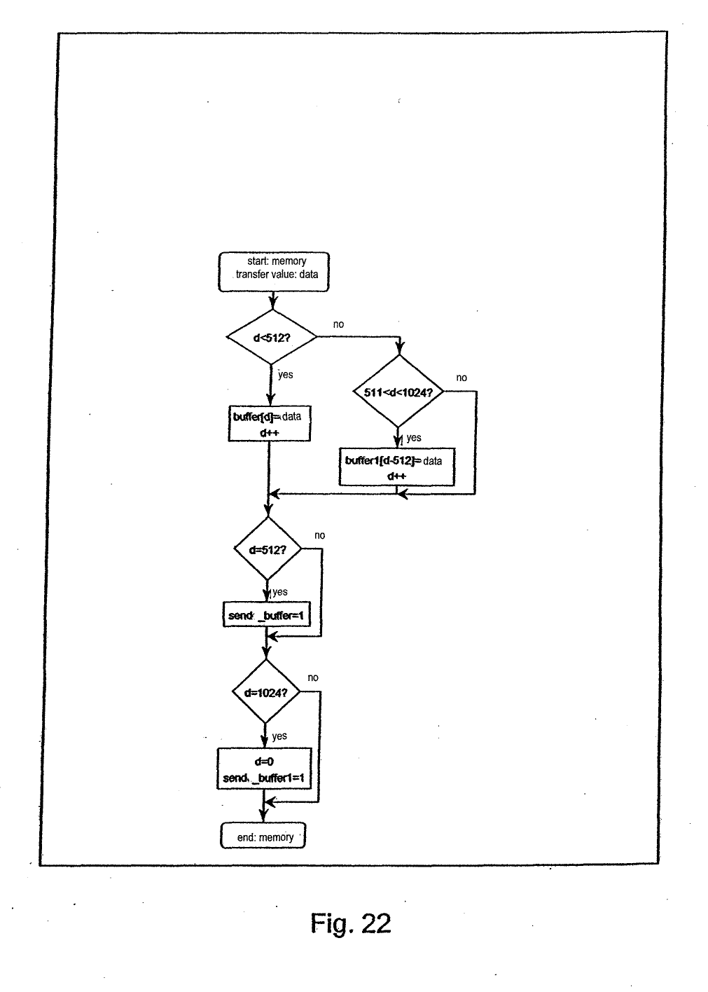

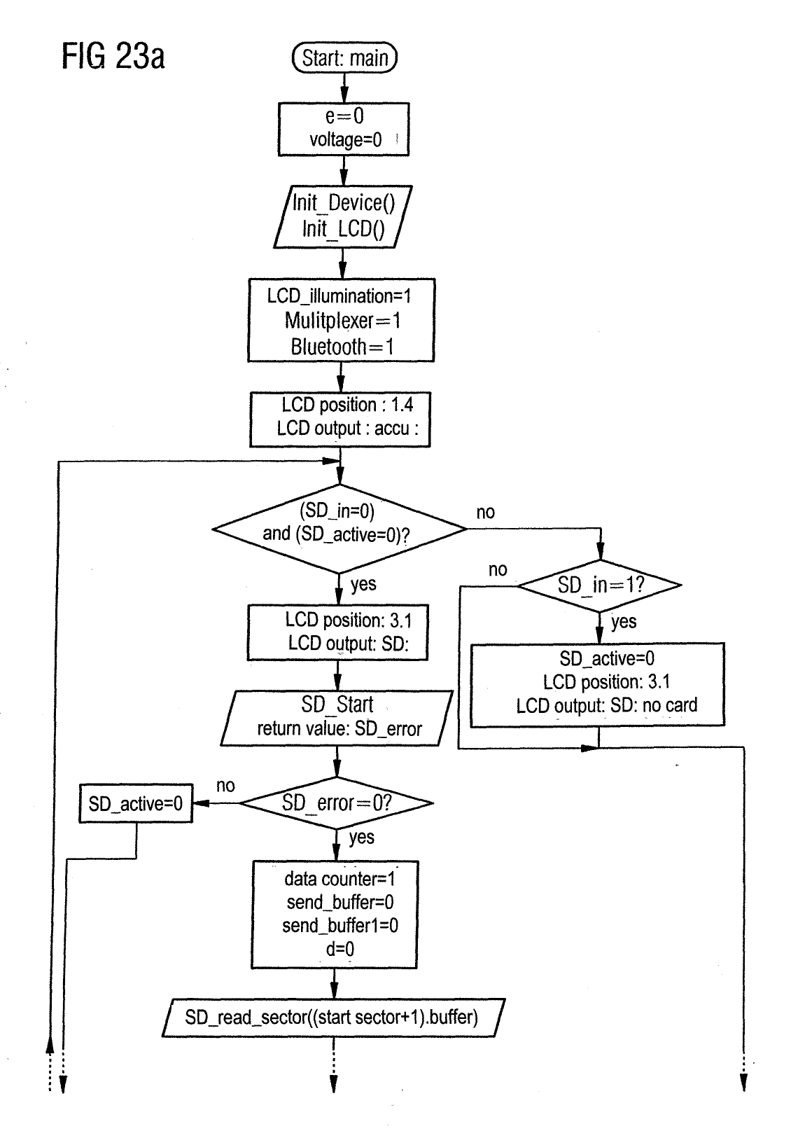

For a better understanding of the complex program flow it is illustrated in a simplified manner in FIG. 16. Only the main program (see FIG. 17) and interrupt service routines (ISR) of the memory unit are illustrated for this purpose. These include the routines designated with “I²C-ISR”, “UART-ISR” (see FIG. 19), and “Timer3-ISR” (see FIG. 18). In addition, the interface to the user software is illustrated in a simplified manner.

In FIG. 16, the program flow of the user software is illustrated in the left column, and that of the memory unit is illustrated in the middle. At the right, the interface to the ISRs is illustrated. The arrows show the flow of data or information between the three modules.

In FIGS. 18 and 19, the program flow of the memory unit is illustrated in the left column, and in the right column there is described where the information comes from/goes to.

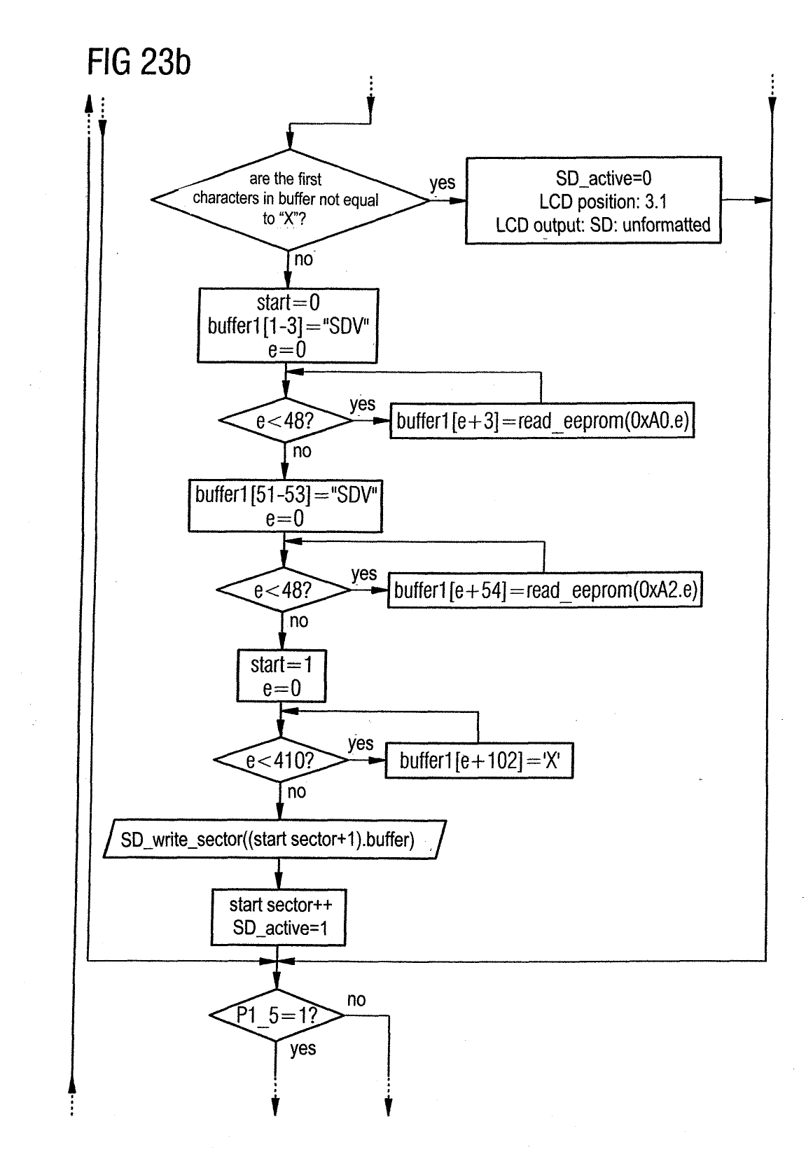

As may be seen in FIG. 20, the embedded software of the memory unit is divided into various header and source code files for the purpose of a better overview. The file “main.c” comprises the main program and the interrupt service routines. In the file C8051F410.h the names of all function registers of the microcontroller used are contained. In the file “init.c” the periphery of the microcontroller is initialized, and in the file “spi.c/i2c.c” the triggering of the SPI/I²C interfaces is contained.

The main program starts with the incorporating of the header files required and with the creating of symbolic names. This is followed by the generating and the initializing of the global variables. Subsequently, the functions and the actual main program as well as the interrupt service routines follow.

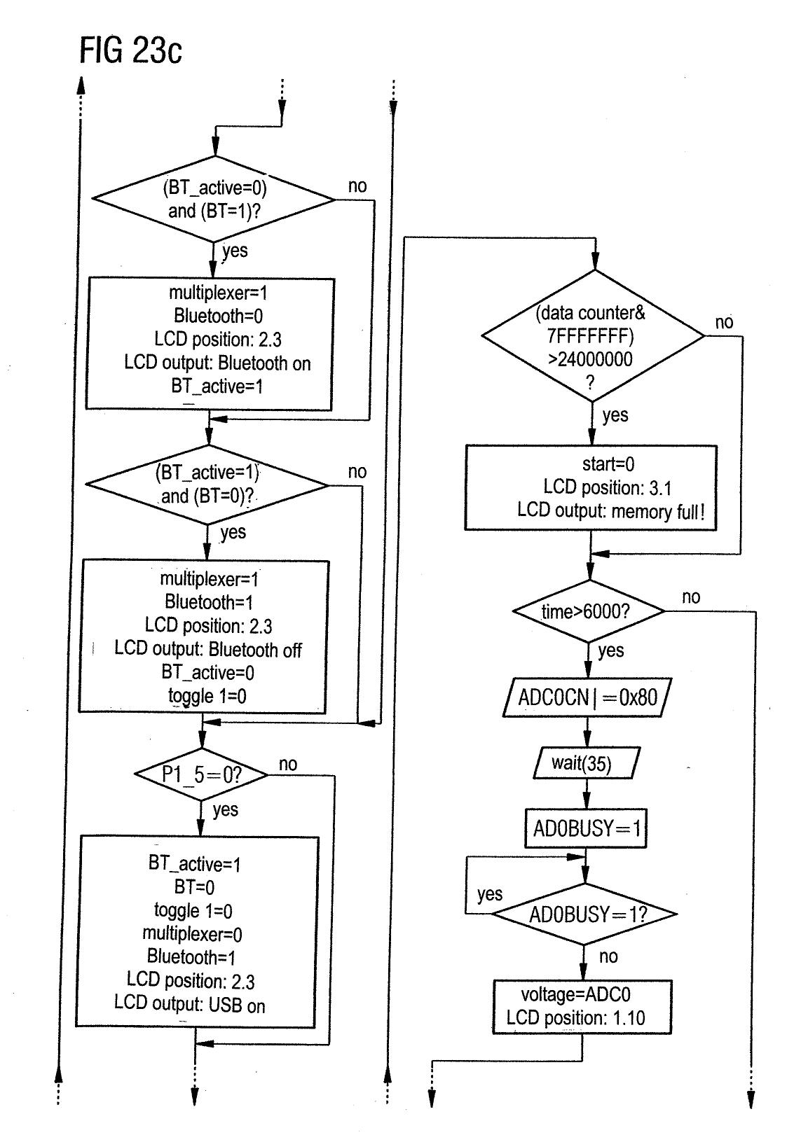

With reference to FIG. 21, the SD_Start function tries to initialize the memory card and searches the start sector. After the initialization of all variables the function “SD_init” tries to initialize the memory card. If this happens without any error, the return value of this function is, for instance, equal to 0. In the case of an error, the function is terminated with an error output on the display. In the case of no error, a start string, e.g. “Epionics”, which characterizes the start of the memory file is searched from a predefined sector, e.g. the sector 500, on the memory card. If it is not found up to a further predefined sector, e.g. the sector 1000, the loop is terminated and an error variable, e.g. the variable “no_start” is set to a particular value that is in the instant case not equal to 0, i.e., for instance, 1. If the variable “no_start” is equal to the determined value, here e.g. 1, the function is terminated at the next request with an error output on the display. If no errors have occurred, the function is terminated with the output “SD okay” on the display.

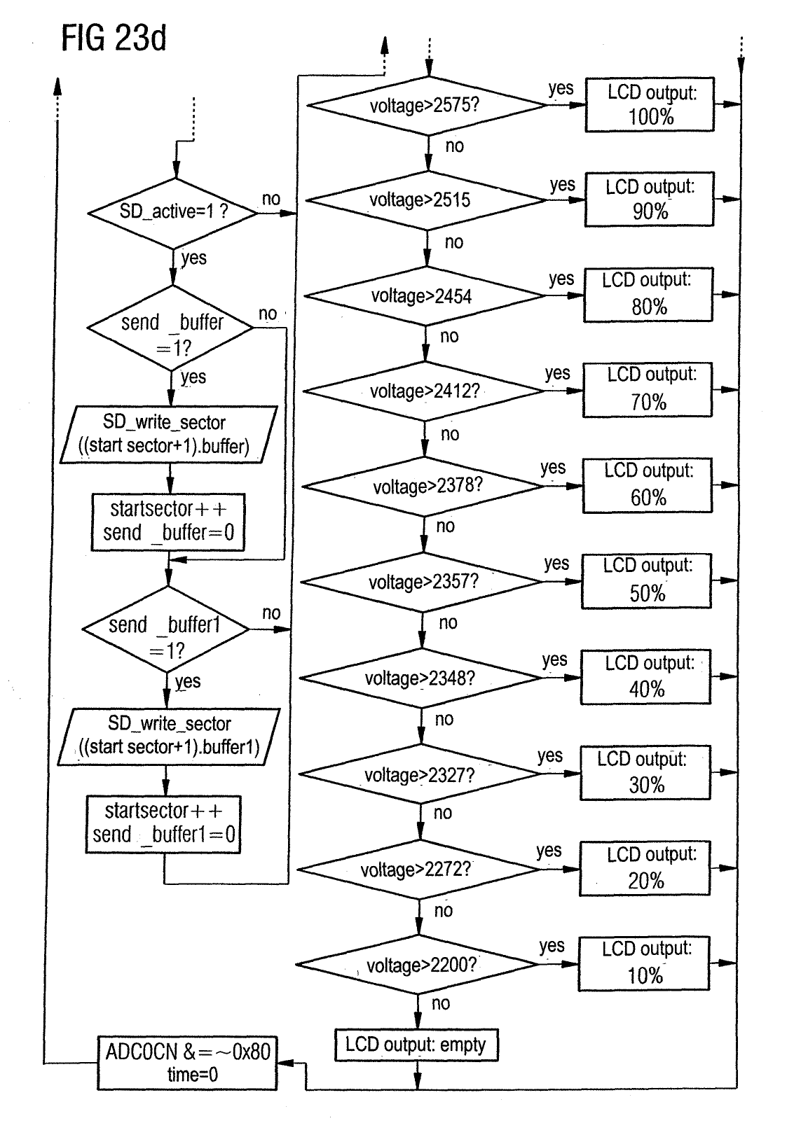

With reference to FIG. 22, the memory function receives the transfer value “data” and inserts it in a memory array. If the memory array is filled with a predetermined amount of data, e.g. 512 bytes, it is written on the memory card. Simultaneously, a second memory array is switched over to in order to accept new data.

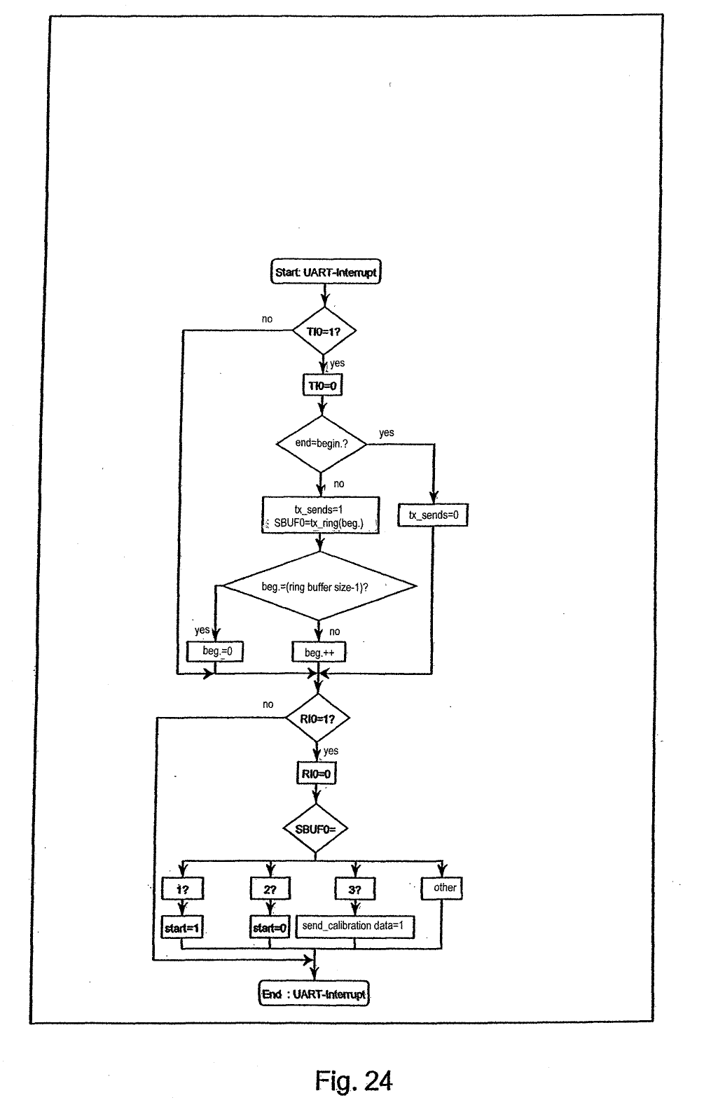

With reference to FIGS. 23a through 23d concurrently, the main program starts with the functions “Init_Device( )” and “Init_LCD( )” in which the microcontroller and the display are initialized. Subsequently the actual endless loop starts. It is examined first whether a memory card has been plugged in. If no memory card has been plugged in, a corresponding message, e.g. “SD: no card” is output on the display. If a memory card is available, it is tried to be initialized. If there were no errors during initialization, the sector after the start sector is read from the memory card. If a predetermined group of characters, e.g. the first five characters of this sector, is not provided with a predefined assignment, e.g. “X”, a corresponding message, e.g. “SD: unformatted” is output on the display. If in the instant example the first five characters are “X”, it is assumed that the memory card is empty. Subsequently, the EEPROMs of both sensor bands are read out and the calibration data and serial numbers contained therein are written on the memory card. Then, the measuring and reading process of the sensor bands is activated. This process runs interrupt-controlled in the background. In the next step there is examined which interface to the user software is activated. If a USB cable is connected, this is the predominant interface. If no USB cable is connected, the user has the possibility of activating the Bluetooth interface by activating the left push-button. The display indicates which interface is activated. Next, there is examined whether one of the two data buffers is full. If this is the case, it is written on the memory card and the start sector is incremented by one. Subsequently, there is examined whether the data counter has arrived at a predetermined number of data sets, e.g. 24 million data sets. In this case, the measuring procedure is stopped and a corresponding error message, e.g. “memory full!” is output on the display. At the end, preferably every minute, the accumulator voltage is measured to implement an approximate accumulator capacitance display. After this point, the endless loop starts afresh.

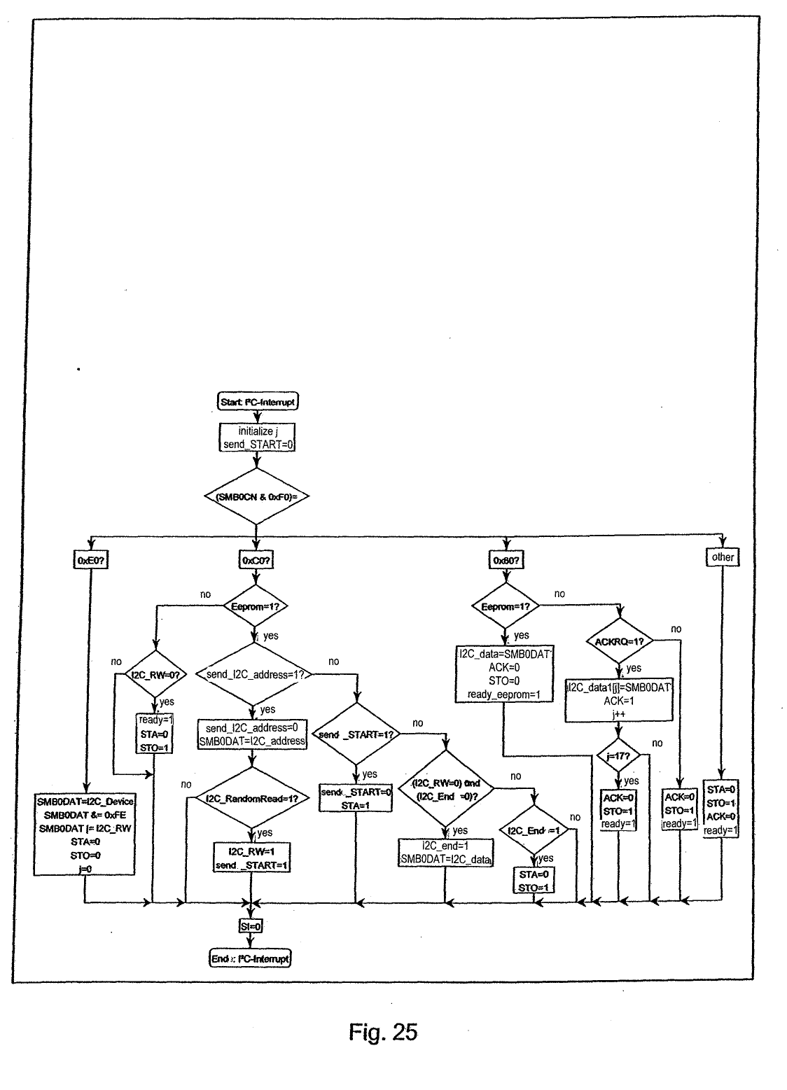

With reference to FIG. 24, the UART interrupt service routine is executed as soon as a character was received or is to be transmitted via the serial interface. The function differentiates between a transmit interrupt (“T10=1”) and a receipt interrupt (“R10=1”). In the case of a transmit interrupt there is examined whether a character is still to he transmitted. If necessary, it will be transmitted. In the case of a receipt interrupt there is examined which character was received and, depending on the received character, the appropriate action, e.g. the staring of the measuring procedure, the stopping of the measuring procedure, the sending of the calibration data, is initiated.

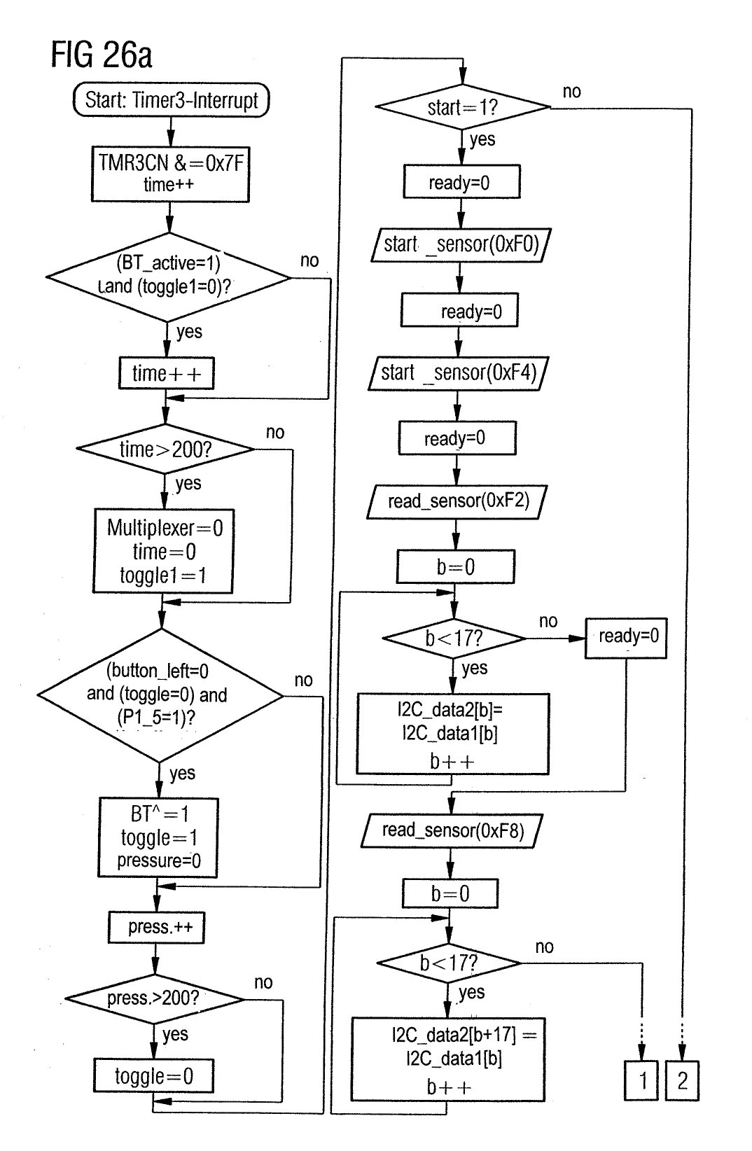

Referring to FIG. 25, in the interrupt service routine for the I²C interface the communication between the memory unit and the sensor bands or EEPROMs, respectively, is controlled. This involves the transmitting of addresses and data and the receiving of data. A differentiation is made between the communication with the EEPROMs and the data exchange to the sensor bands.

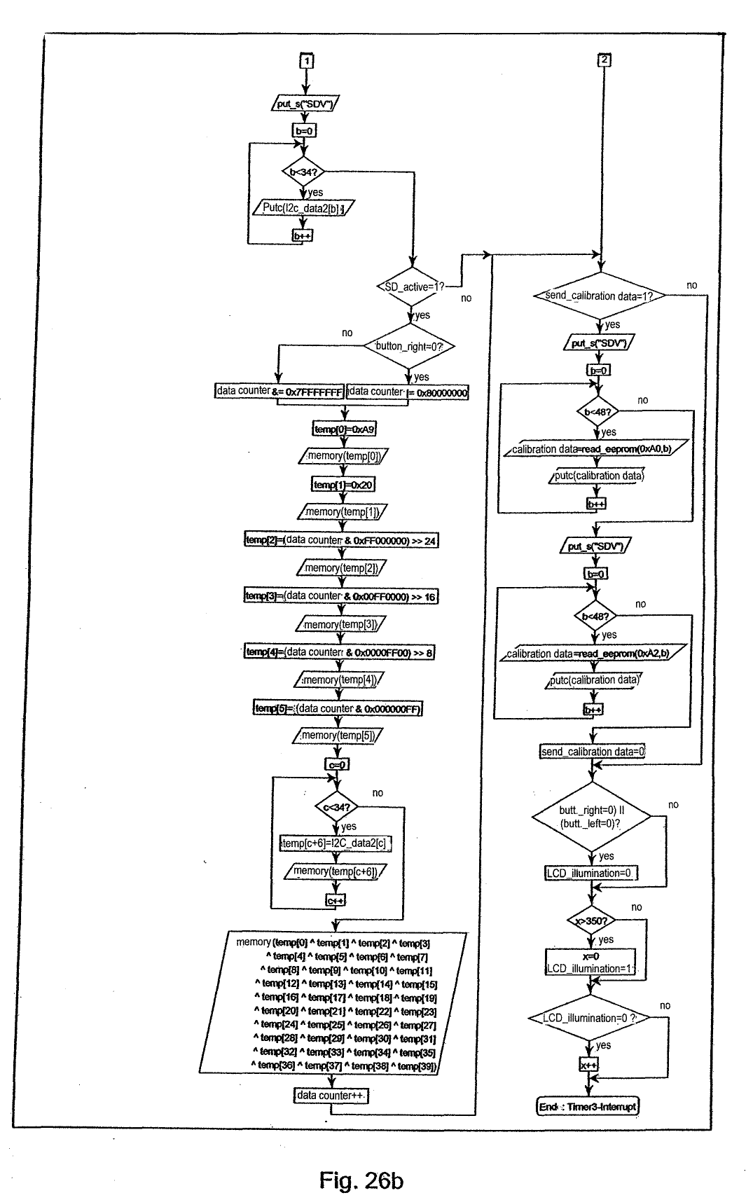

With reference to FIGS. 26a and 26b, the timer3_isr interrupt routine is shown. A timer, here “Timer3”, is adjusted such that its interrupt service routine is executed at predetermined intervals, e.g. every 10 ms. Here, the push-button control, the switching on and off of the display background illumination, the transmitting of the calibration data, and the starting and reading out of the sensor bands are implemented.

An exemplary selection of the functions or objects of the file “init.c” include the following:
Deactivation of the microcontroller-internal voltage regulator.
Defining of all inputs and outputs.
An oscillator initialization that activates the external oscillator and waits until it runs soundly.
Initialization of several timers, e.g.:
   Timer0 is initialized in the 8 bit autoreload mode for the I²C bus. Its clock is 360 kHz in this embodiment, which corresponds to an I²C clock of 120 kHz.
   Timer1 is initialized in the 8 bit autoreload mode for the UART interface. Its clock is 230 kHz in this embodiment, which corresponds to an UART baud rate of 115200 bits per second.
   Timer3 is initialized in the 16 bit autoreload mode. Its clock is exactly 100 Hz in this embodiment and is used as a time basis for the measuring cycles.
I²C initialization: the I²C bus is activated as master, the clock source is selected, and the automatic timeout detection is activated.
Setting of the serial interface to 8 bit data length by means of a start and stop bit, and activation of the receipt.
Initialization of the SPI interface in the 4-wire single master mode with a clock of, for instance, 1 MHz.
Clocking of the A/D converter with the system clock and accepting of a measurement value.
Interrupts are released globally and activated for UART, I²C, and Timer3. Moreover, I²C and UART are given a high interrupt priority.
Deactivation of the voltage monitor of the microcontroller and of the watchdog timer.
Waiting loop function.
Providing of a ring buffer of 110 bytes which transfers data to the serial interface, wherein the data are transmitted as individual byte values or strings.

An exemplary selection of the functions or objects, respectively, of the file “spi.c” include the following:
Transmission of a transferred byte value via the SPI bus.
Reading of a byte value from the SPI bus.
Sending of a command to the memory card and receiving of the response of the memory card.
Initializing of the memory card with reduced SPI clock.
Reading of data blocks from the memory card.
Reading of a sector, e.g. 512 bytes, from the memory card according to transferred memory address and memory variable.
Writing of a data block, e.g. 512 bytes, on the memory card according to transferred memory address.
Sending of a transferred command to the display.
Initializing of the display.
Sending of an individual character to the display.
Sending of a string to the display.
Setting of the cursor to a particular position on the display.

An exemplary selection of the functions or objects, respectively, of the file “i2c.c” include the following:
Writing on the EEPROMs on the sensor bands according to the transferred EEPROM address, memory address, and data value.
Reading out the EEPROMs on the sensor bands according to the transferred EEPROM address and memory address.
Starting the measuring procedure on the sensor band with the transferred address.
Starting the data transmission from the sensor band with the transferred address.

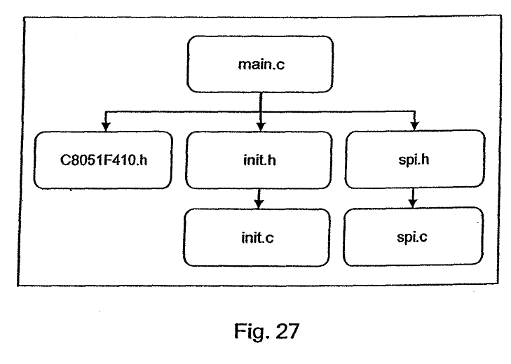

As may be seen in FIG. 27, the embedded software of the sensor band is divided into different header and source code files for the purpose of a better overview. The file main.c contains the main program and the interrupt service routines. In the file C8051F410.h there are contained the names of all function registers of the μC used. In the init.c the periphery of the μC is initialized, and in the spi.c there is contained the control of the SPI interface.

Since two sensor bands are available at a bus, it is necessary to give the left sensor band an address that differs from that of the right sensor band. Per sensor band there is one address available for starting the measurement and one address for sending the measurement values. Due to these different addresses a differentiation is possible.

The main program starts with the incorporating of the required header files and the creating of symbolic names. Then, the generation and initialization of the global variables are performed. Subsequently, the actual main program and the interrupt service routines follow.

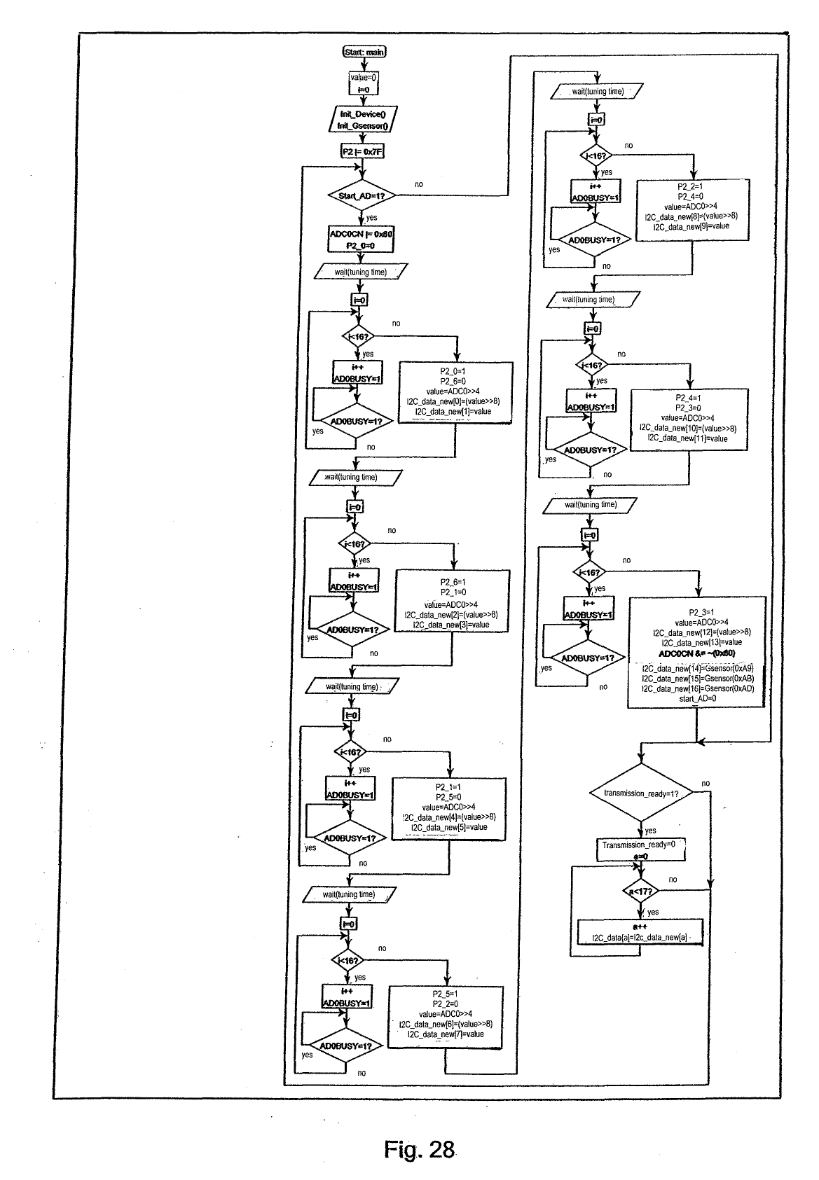

Referring now to FIG. 28, the main program starts with the functions “Init_Device( )” and “Init_Gsensor( )” in which the μC and the position sensor are initialized. Then, the FETs for all measuring bridges are deactivated. Subsequently, the actual endless loop starts. In it, the variable “Start_AD” is queried. If it is equal to a first predetermined value, e.g. 0, the program is in an “idle” state. Only if this variable is equal to a further predetermined value, e.g. 1, does the reading out and digitizing of the measurement values start. If this is the case, the A/D converter and the first measuring bridge are activated. In order that the system can tune there is waited for a short time before the A/D converter starts the measurement. For averaging, a number of values, e.g. 16 values, are read in and the average value is calculated and stored. Then, this measuring bridge is deactivated, the next one is activated, and the measuring and storing procedure starts again until all measuring bridges were measured. Finally, the A/D converter is deactivated and the position sensor is read out. The three measurement values for each dimension are also stored. Subsequently, there is examined whether the last data were already retrieved from the memory unit. If this is the case, the new measurement values are written into the data array for new transmission.

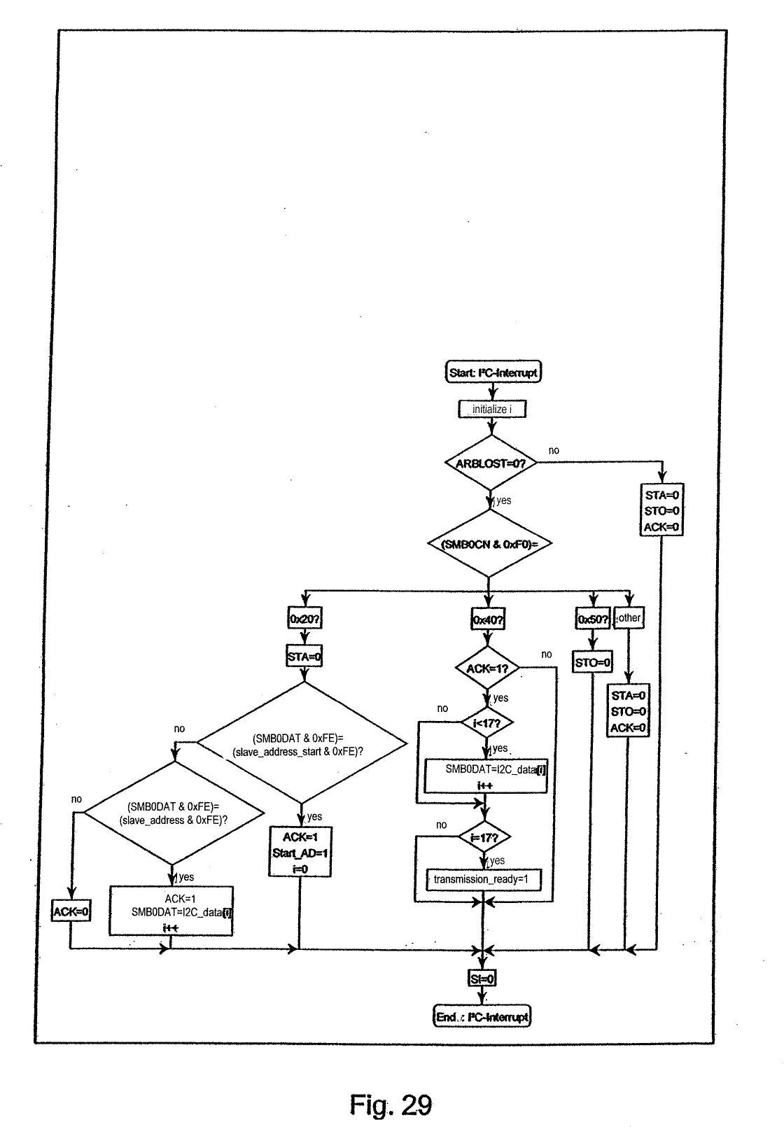

With reference to FIG. 29, to the interrupt service routine for the I²C interface the communication between the sensor band and the memory unit is implemented. The sensor band operates as a slave, i.e. only queries from the master are responded to. If the sensor band receives the address for reading out the sensors, the measuring procedure is started. When the address for sending the measurement values is received, the data transmission to the memory unit starts.

An exemplary selection of the functions or objects, respectively, of the file “init.c” includes the following:
Deactivation of the microcontroller-internal voltage regulator.
Activation of the external reference voltage source for the A/D converter to achieve higher accuracy.
Defining of all inputs and outputs.
Initializing of “Timer0” in the 8 bit autoreload mode for the I²C bus, wherein its clock is approx. 360 kHz, which corresponds to an I²C clock of approx. 120 kHz.
I²C initialization: the I²C bus is activated and the clock source is selected.
Initializing of the SPI interface in the 4-wire single master mode, wherein the clock is set to approx. 3 MHz.
Globally releasing the interrupt routines and activate them for I²C.
Deactivation of the voltage monitor of the μC and of the watchdog timer.
Waiting loop function.

An exemplary selection of the functions or objects, respectively, of the file “spi.c” includes the following:
Transmitting of a transferred byte value via the SPI bus.
Reading of a byte value from the SPI bus.
Adjusting the position sensor to a predetermined measurement range, e.g. ±2G (wherein “G” stands for the gravitational acceleration) and to a predetermined sampling rate of e.g. 100 Hz.
Starting of the sensor.
Addressing the position sensor with the transferred address, wherein the measurement value is returned as a response.

For calibrating the sensors, a separate embedded software is used in the memory unit. It stores the calibration data in the EEPROM of each sensor and examines whether the data were stored accurately. The calibration data are expediently entered in the embedded software by hand for each sensor band, and each sensor band is individually programmed therewith.

The user software is divided into two parts. The “live software” receives the data directly from the Bluetooth interface of the memory unit and visualizes them immediately. The “analysis software” is capable of loading previously stored data sets from the hard disk and represent them.

In the following, one preferred embodiment each of the two parts of the user software, the “live software” and the “analysis software” will be described in more detail. These embodiments only serve for explanation purposes and have a purely exemplary character. It is noted that the scope of protection of the present invention is not to be defined or to be restricted on the basis of the features of these embodiments.

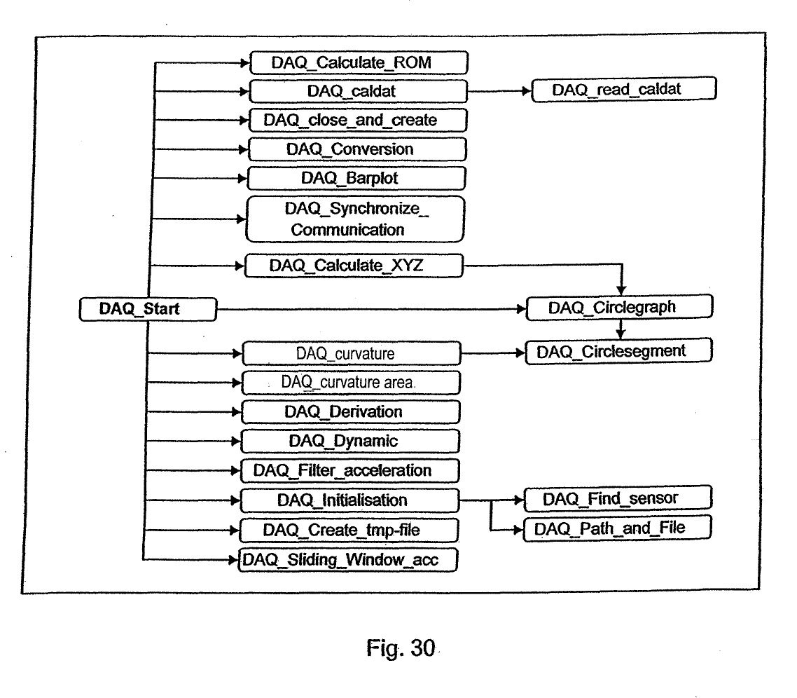

FIG. 30 shows an overview of the subprograms used in the live software. Most subprograms are called up directly from the main program DAQ_Start. In some cases, further branching occurs.

DAQ_{—}0_Start forms the main program. Here, all the other functions are triggered and all the results and visualizations are brought together.
Initialize sensor bands (see DAQ_Initialization)
Read in calibration data (see DAQ_Caldat)
Synchronize communication with the memory unit (see DAQ_Synchronize_Communication)
Read in a data set from the serial interface
Convert data set by means of the calibration data to angles (see DAQ_Caldat)

DAQ_read_caldat is the subprogram for reading in and storing the calibration data. The subprogram calls up the function DAQ_read_caldat and tries to read out the calibration data of the connected sensor bands and to convert them by means of the formula:

Calibration data=input value*2.5V/4096.

In the case of success the subprogram is terminated and the calibration data are returned. If the reading-out should not be successful, the subprogram closes the comport and connects it anew. Thus, the connection is initialized anew. Then, there is another effort of reading in the calibration data. The procedure is repeated maximally three times. In the case of success the calibration data are returned, in the case of failure just an empty field is returned.

The subprogram DAQ_Initialization searches comports at which measuring devices are sending. To this end, the subprogram DAQ_find_sensor is called up. Subsequently, the user may chose a comport and determine the memory location and the name of the memory file via the inputs “Name,” “Filename,” and “Path.” In correspondence with the information, folders and files are subsequently established with the subprogram DAQ_Path_and_File.

The subprogram DAQ_find_sensor searches available comports at the computer. Then, every detected comport is opened and a predetermined number of characters are read in. If they contain a particular string, e.g. “SDV,” the comport is added to the output list. If the output variable is still empty after the test of all the comports, the subprogram is repeated again.

The function DAQ_Path_and_File establishes two subdirectories in the input folder. Firstly a directory with the name of the patient and secondly a directory with the current date.

The function DAQ_Calculate_ROM examines whether the current angles lie within the previous maxima. If the maxima should be exceeded, the output values are overwritten with the new maxima. Moreover, the function calculates the fields with data necessary for the ROM display.

The function DAQ_Synchronize_Communication examines whether the communication with the memory unit is synchronized. To this end, it reads in a complete data set and examines whether the first three bytes correspond to a particular string, e.g. “SDV”. If this is the case, the communication is synchronous and the subprogram is left. Otherwise, one byte is read in and the function starts again. Thus, the read-in data sets are always shifted by one byte until the communication is synchronous.

The DAQ_close_and_create subprogram closes the current memory file and then tries to create a new memory file in the directory. Subsequently, the new memory path is returned.

The DAQ_Conversion subprogram reads in the data sets as strings and converts them by means of the calibration data to angles or accelerations. Conversion formulae of the angles:

voltage=[highbyte+(256*lowbyte)]*2.5V/4096

angle_{greater0}=[12.5°/(calibration value_{12.5°}−calibration value_{0°})]*(voltage−calibration value_{0°})

angleₛₘₐₗₗₑᵣ₀==−12.5°+[12.5°/(calibration value_{0°}−calibration value_{12.5°})]*(voltage−calibration value_{-12.5°})

Conversion formulae of the accelerations:

two's complement=256+byte for byte>127

two's complement=256+byte for byte<=127

acceleration=(two's complement/255)*2*maximum acceleration range

The DAQ_Barplot subprogram converts the input data such that they can be illustrated as a bar plot in an XY plot.

The DAQ_Calculate_XYZ subprogram receives the current angles as an input and returns the Cartesian coordinates of the segments of a circle. To this end, first of all the angles of the right sensor are separated from that of the left sensor, and then they are transmitted to the subprogram DAQ_Circlegraph. Moreover, the values obtained are given a suitable form.

This DAQ_Circlegraph subprogram splits the angles of a sensor, has the curve progressions of the individual segments calculated, and brings the results together to a plot. For calculating the individual segments, the function DAQ_Circlesegment is called up.

Initially, the DAQ_Circlesegement subprogram examines whether the current angle is equal to zero. Since zero is an asymptote (radius infinite) for angle calculation, the input angle is set to 0.0001° in this case. The circle segment is divided into 51 sections. It is calculated by the following formula:

angle section in radian=((input)angle/180°)*π)/51.

In parallel, the radius for circle calculation is calculated from the angle.

radius=(50 mm*360°)/(angle*2π)

Now, the circle formulae, which follows, are used:

X=radius*cos(angle)

Y=radius*sin(angle)

The angle is incremented by one angle section with every loop cycle. Moreover, the offset angle is added to the angle with every loop cycle. After the last loop cycle the current angle is transferred. The entire circle segment is now shifted by a predetermined value (“offset”). Thus, the current circle segment starts exactly at the end of the previous segment. The last coordinates of the current circle segment are transferred to the variables and are available as starting points for the next segment.

The DAQ_Curvature subprogram uses the function DAQ_Circlesegment for the calculation of the progression of a sensor. In contrast to DAQ_Circlegraph all the circle segments are comprised in a plot (curvature). The end point of the curvature is returned by the variables. Thus, the angle for turning the curvature representation is determined.

The DAQ_curvature_area subprogram searches the zero crossing of the Y coordinates and divides the graph into two areas in this point. Subsequently, the surface areas of both areas are calculated with

$$surface area = {\sum }_{i = 1}^{n - 1} \begin{matrix}\{ ( x [ i + 1 ] - x [ i ] ) \cdot y [ i + 1 ] \} - \\ \{ \frac{( x [ i + 1 ] - x [ i ] ) - ( y [ i + 1 ] - y [ i ] )}{2} \}\end{matrix}$$

with n=number of curve points
   If no zero crossing exists, only one area is calculated.

In the DAQ_Derivation subprogram, rate and acceleration values are calculated.

The subprogram DAQ_Dynamic calculates the dynamic plot by means of the below-mentioned formulae from the input angles.

$$Xcoordinate [ i ] = {\sum }_{n = 1}^{7} input {angle}_{segment n} [ i ]$$

$$Ycoordinate [ i ] = 10 \cdot ( input angle [ i ] - input angle [ i + 10 ] )$$

The subprogram DAQ_filter_acceleration processes the respectively last 30 data points of every acceleration vector. These are added and divided by 30.

$$acceleration value [ i ] = \frac{{\sum }_{n = 1}^{30} acceleration value [ i - n ]}{30}$$

Thus, a smoothing of the acceleration values is achieved. Peaks that were, for instance, produced by walking are suppressed.

The subprogram DAQ_Sliding_window_acc works similarly to DAQ_filter_acceleration. However, the formula, which follows, is not just used for the last 30 data points, but sequentially for the entire field:

$$acceleration value [ i ] = \frac{{\sum }_{n = 1}^{30} acceleration value [ i - n ]}{30}$$

The output field thus becomes 30 values shorter than the input field.

The subprogram DAQ_create_tmp-file serves to prevent an accidental overwriting of measured data. After a measurement was terminated by “storing”, the subprogram is called up and adds the string “_new” to the file name of the last memory tile. If also this file name should already exist and the user should refuse the overwriting of the file, the adding is repeated until a new file is found.

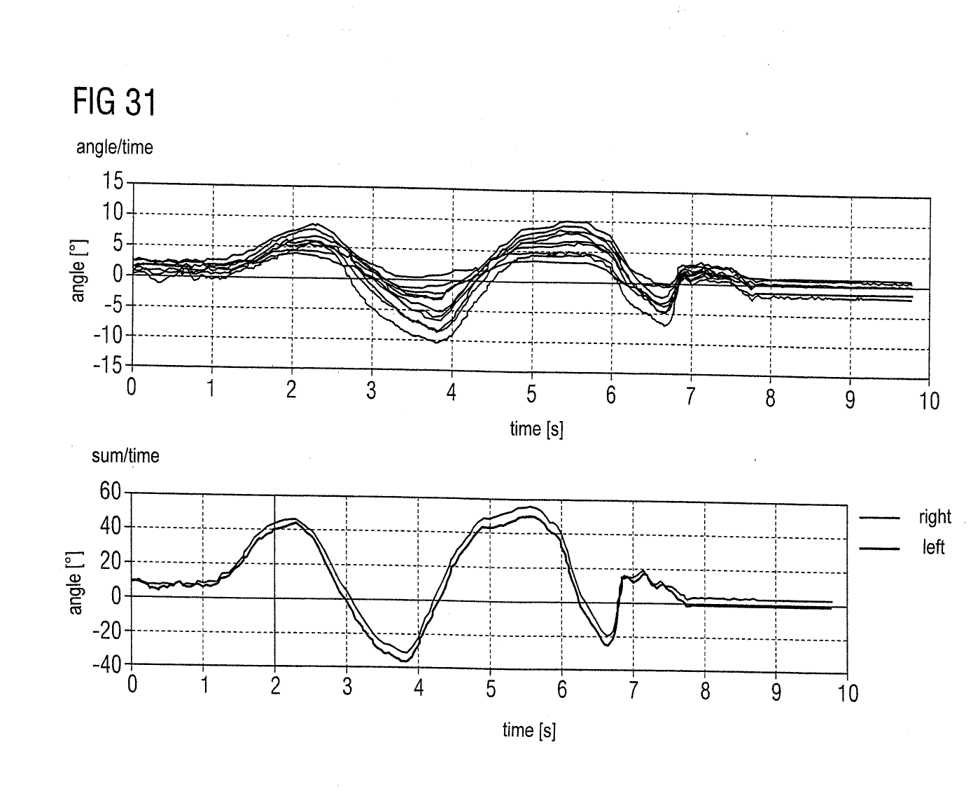

FIG. 31 shows a first raw data plot in which the angles of the individual segments of the right and left sensors are illustrated over time. In addition to the individual channels, the angle sums of the right and left sensors are also illustrated.

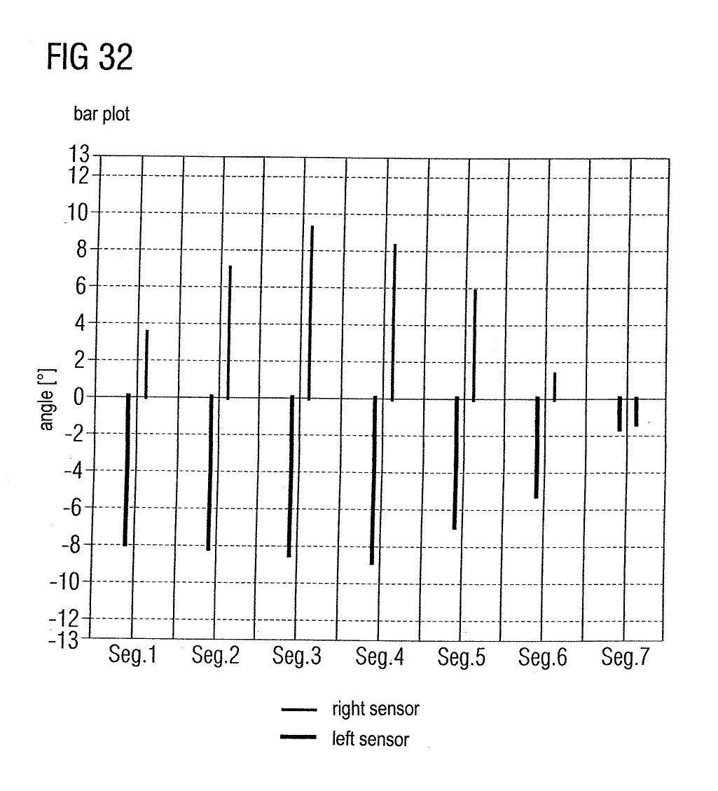

FIG. 32 shows a second raw data plot in which the current angles are illustrated by segments. There is a differentiation between the right and the left sensor.

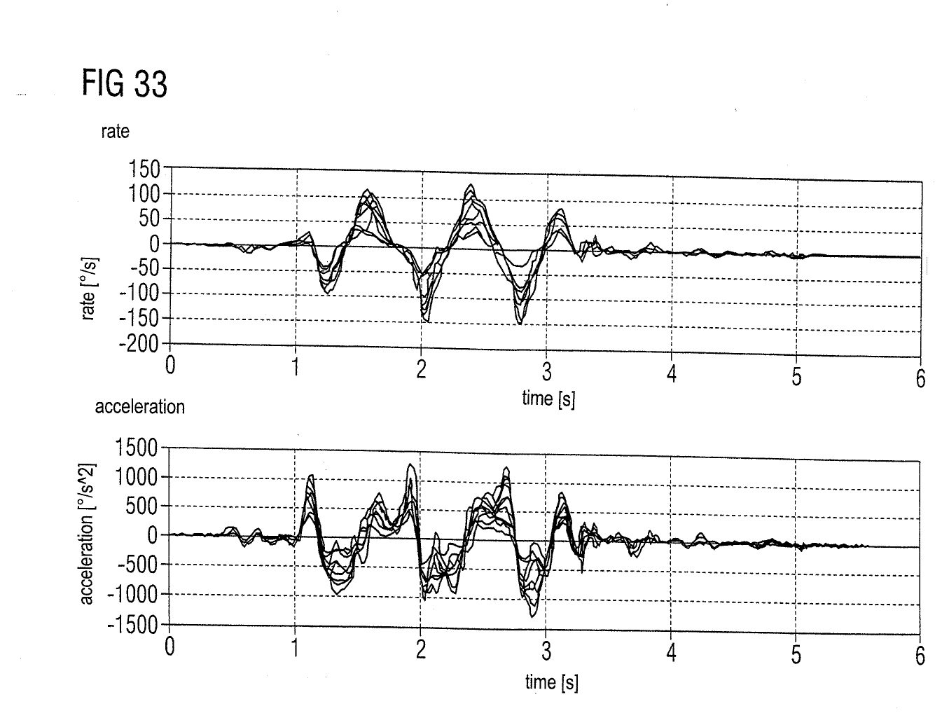

FIG. 33 shows two plots in which the angle rate and the angle acceleration are each plotted over time. The data are determined by discrete derivation with a distance of for instance, 10 data points.

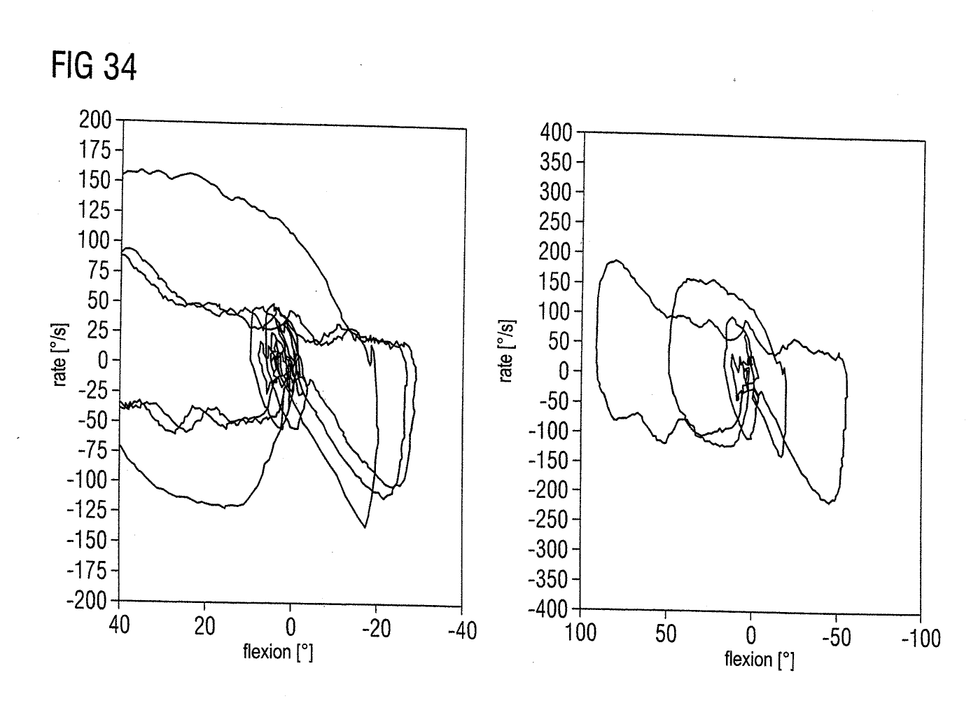

FIG. 34 shows two dynamic plots in which the angle rate is plotted over the angle sum. In the left diagram the sums of the left and right sensor bands are to be seen. In the right plot both sensor bands are added and viewed as a whole.

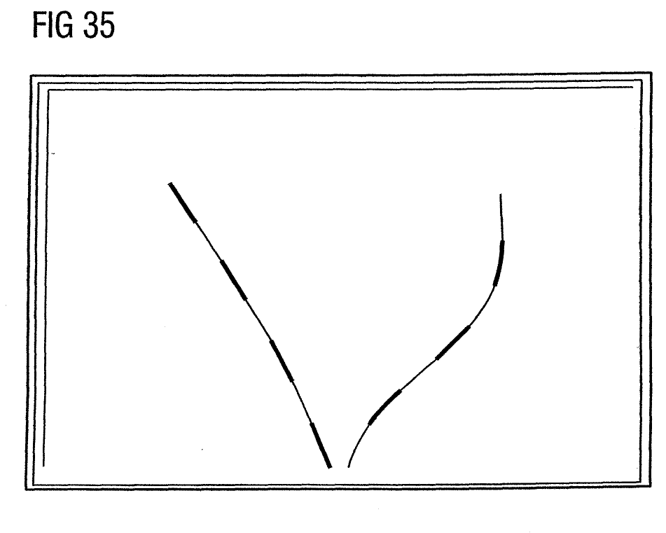

FIG. 35 shows a bar plot in which the current angles and accelerations of the position sensor are processed. The X and the Z axes of the position sensors determine the start vector of the sensor bands. The angles determine the progression. There is differentiated between the right and the left sensor bands.

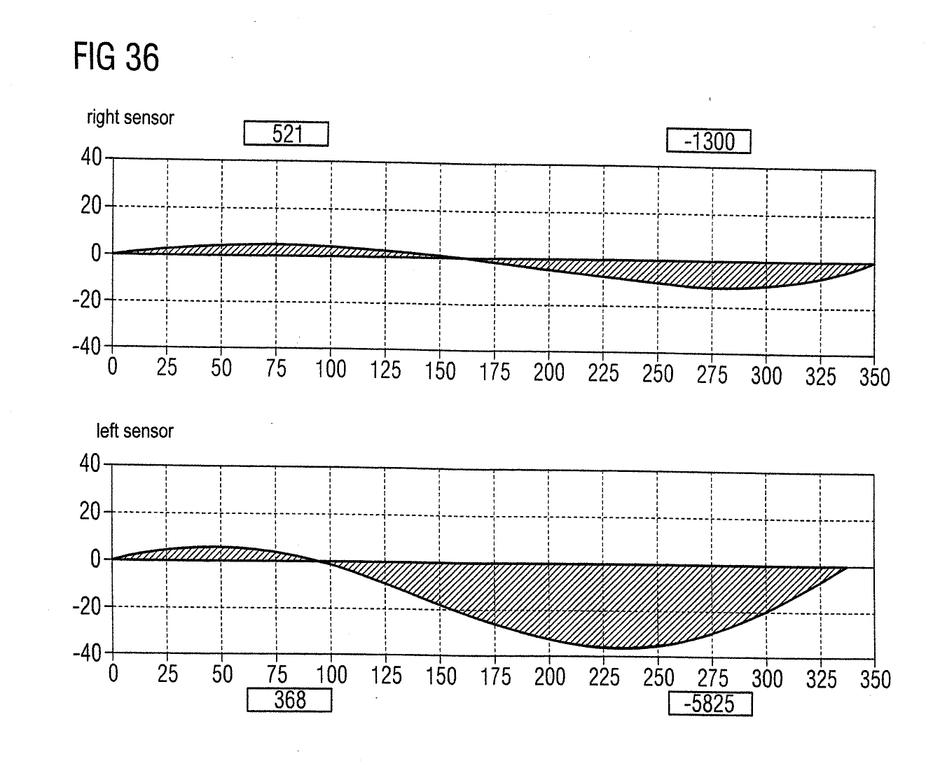

FIG. 36 shows illustrations of the curvature areas for the right and the left sensors separately, in which the current bending angles are visualized. The start and end points of the sensor band are turned in one plane for this purpose. Subsequently, the areas separated by the zero crossing are calculated and displayed.

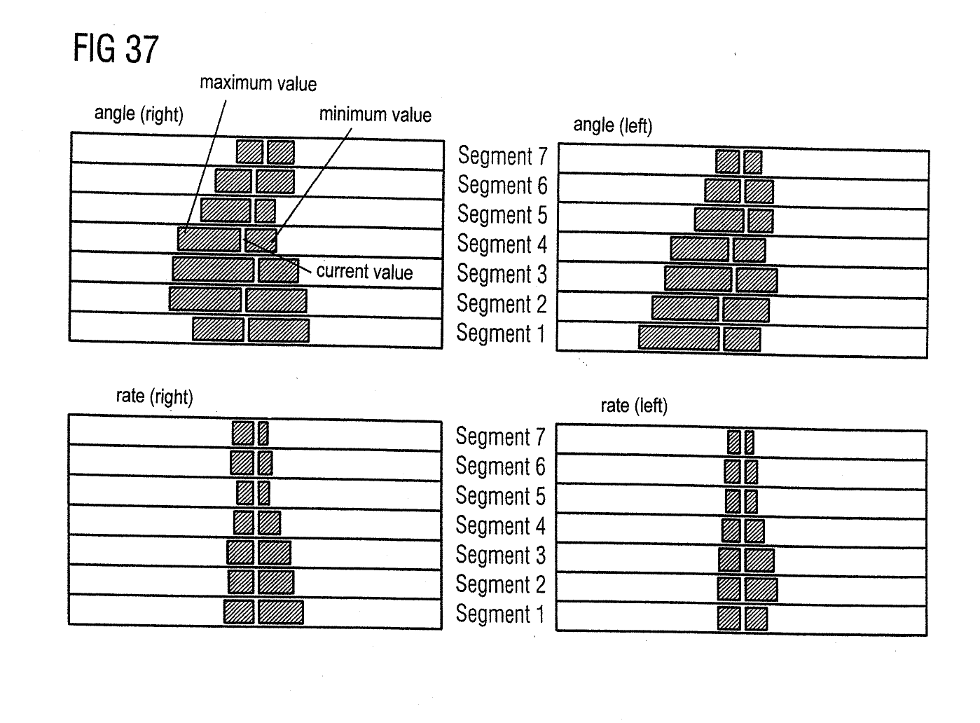

FIG. 37 shows a so-called “range of motion” illustration in which the current angles and angle rates (each for the right and the left sensors separately) are illustrated and their maximum and minimum values are detected.

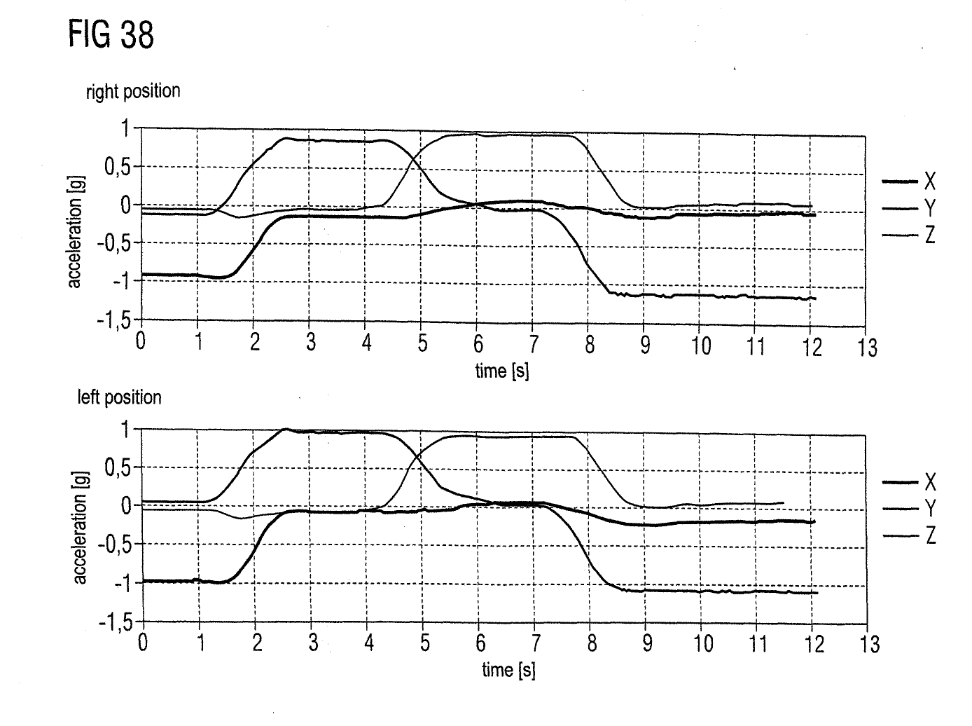

FIG. 38 shows a raw data illustration of the position sensor in which the results of the position sensors of both sensor bands are illustrated. The three space coordinates X, Y, and Z are differentiated. The measurement values are strongly filtered since information about the position is desired.

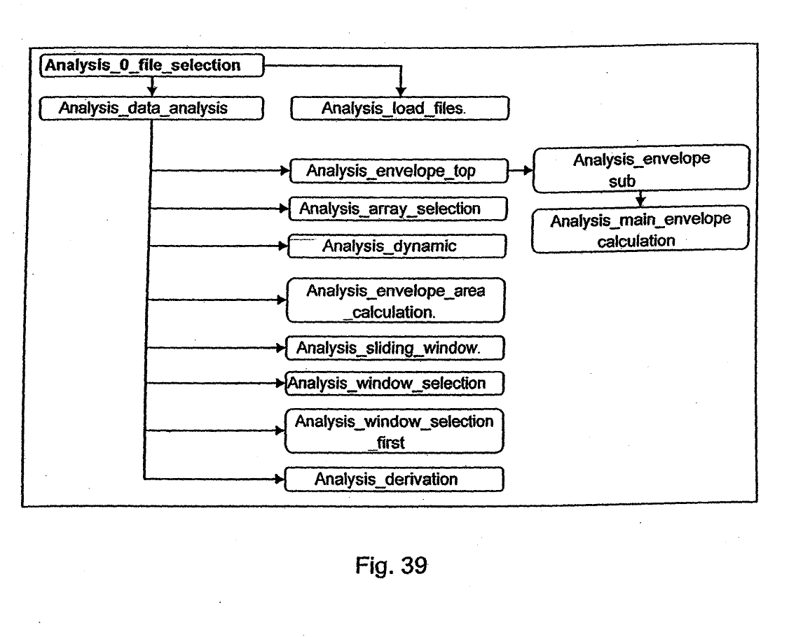

FIG. 39 shows an overview of the subprograms used in the analysis software.

The Analysis_{—}0_file_selection program serves as a starting point for the data analysis. Here, the measurement files to be analyzed are selected, loaded, and the data analysis is started.

In the Analysis_load_files program, the position field transferred contains information about the measurement files for the data analysis. These are loaded and adhered to one another by the function. Subsequently, the field is returned.

The subprogram Analysis_data_analysis forms the main function for data analysis. The different forms of visualization are selected therein. For this purpose, the data are sorted in angles and accelerations of the position sensor, smoothed on demand, and subsequently visualized. With the forms of visualization for the bending angles the user than has the additional possibility of showing or hiding individual sensor segments in the illustration.

By means of the measured angle data the subprogram Analysis_window_selection_first determines the maximum values and the minimum values of the slide controls characterized by the references. This subprogram is called up only once at the beginning of “Analysis_data_analysis.”

The subprogram Analysis_window_selection cuts the desired time range out of the entire measured data in corresponding with the position of the slide control and returns them to “Analysis_data_analysis” for illustration. Thus, a zoom to the desired measured data is achieved.

In the subprogram Analysis_sliding_window, the following formula is used to average the input data:

$$output value [ i ] = \frac{{\sum }_{n = 1}^{Window\_in} input value [ i + n ]}{Window\_in}$$

In so doing, the field is shortened by Window_in positions.

The subprogram Analysis_derivation (see DAQ_Curvature area) searches the zero crossing of the Y coordinates and divides the graph in two areas in this point. Subsequently, the surface areas of both areas are calculated with

$$surface area = {\sum }_{i = 1}^{n - 1} \begin{matrix}\{ ( x [ i + 1 ] - x [ i ] ) \cdot y [ i + 1 ] \} - \\ \{ \frac{( x [ i + 1 ] - x [ i ] ) - ( y [ i + 1 ] - y [ i ] )}{2} \}\end{matrix}$$

with n=number of curve points
   If no zero crossing exists, only one area is calculated.

The subprogram Analysis_dynamic can be understood with reference to the subprogram DAQ_Dynamic.

The Analysis_array_selection function cuts out selected sensor channels and returns them to “Analysis_data_analysis.” Thus, the user may restrict calculations and visualizations to selected sensor regions.

In the Analysis_envelope_area_calculation subprogram, the following formula is used to calculate the integral of the envelope:

$$area = {\sum }_{i = 1}^{n - 1} \begin{matrix}\{ ( x [ i + 1 ] - x [ i ] ) \cdot y [ i + 1 ] \} - \\ \frac{( y [ i + 1 ] - y [ i ] ) \cdot ( x [ i + 1 ] - x [ i ] )}{2}\end{matrix}$$

for n=length of envelope
   The calculated value is not the exact integral, but only the approximation via the trapezoidal rule.)

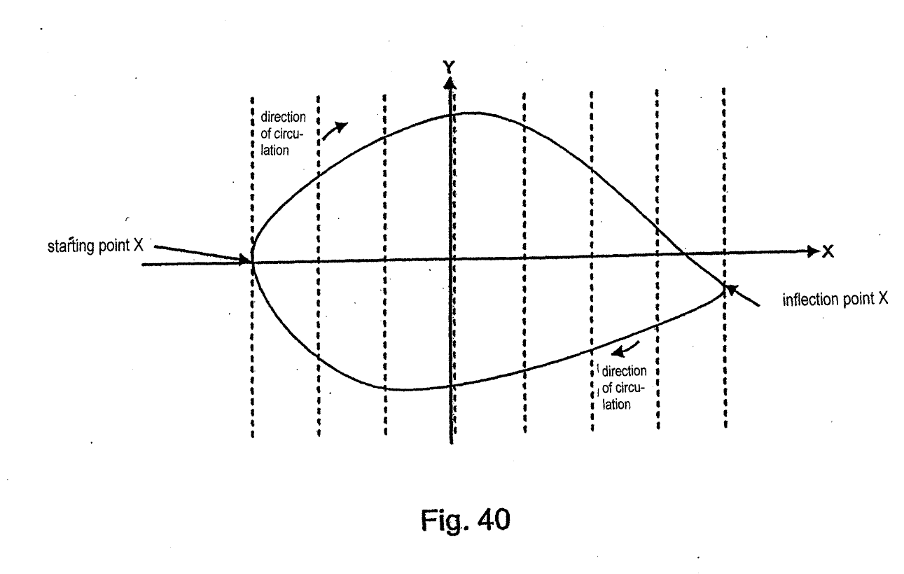

In the subprogram Analysis_envelope_top, the envelope of a dynamic plot (see FIG. 40) is to be calculated. To this end, n intervals are calculated between the starting point X and the inflection point X. The envelope is composed of the following points.
Starting point X
The maximum point of each interval beginning at the starting point in the direction of circulation.
Inflection point X
The minimum point of each interval beginning at the inflection point in the direction of circulation.
Starting point for closing the envelope.

The maximum and minimum points are to be calculated in parallel. This subprogram forms the first instance of envelope calculation. Here, the minima and maxima of the X and Y coordinates and the interval width are determined. Subsequently, the subordinate subprogram “Analysis_envelope_sub” is called up. With the values retrieved from there, the envelope is then composed.

The subprogram Analysis_envelope_sub successively calculates the current interval boundaries in X direction and then calls up the subprogram “Analysis_main_envelope_calculation.” This subprogram supplies the coordinates of all points that lie in the current interval. Subsequently, the maximum or minimum values are searched within these coordinates and are transferred to “Analysis_envelope_top.”

The subprogram Analysis_main_envelope_calculation searches all values in the current max and min interval and returns them.

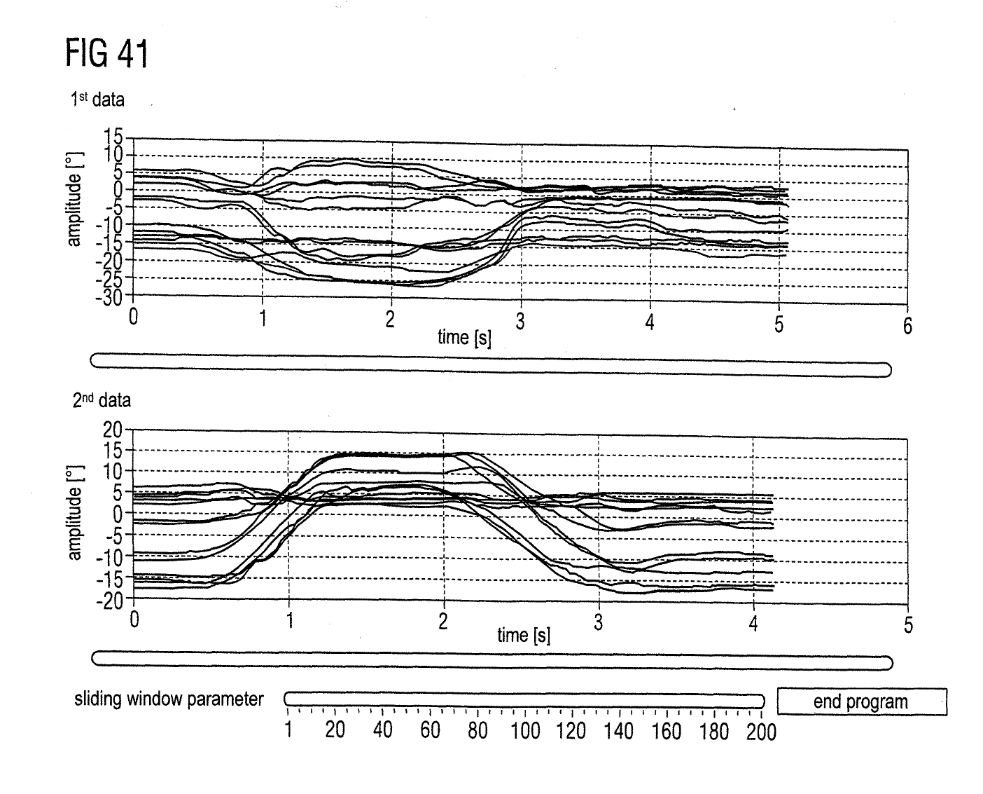

FIG. 41 shows a raw data illustration in the analysis software in which the angle is plotted over time. A time range may be selected via the shift controls under the plots. Thus, it is possible for the user to view small time periods in a zoomed manner. Via the “sliding window parameter” it is possible to additionally smooth the raw data. The illustrated sensor segments may be selected individually.

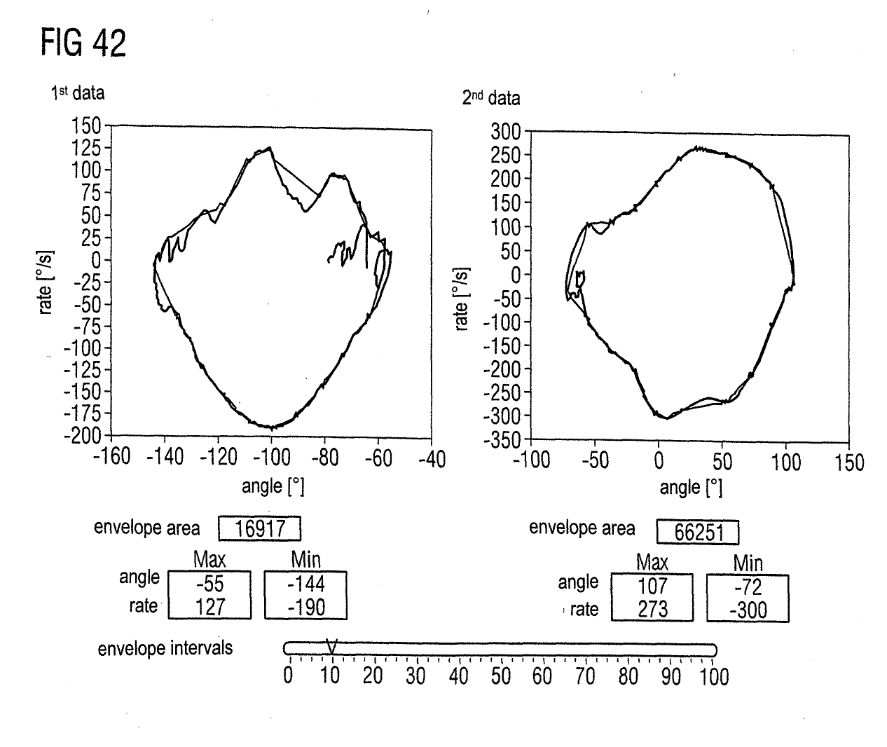

FIG. 42 shows dynamic plots with an angle over rate illustration of the raw data. As parameters, the user here obtains the maximal and minimal angles and rates and moreover the surface area of the envelope. The number of intervals for envelope calculation may be adjusted via “envelope intervals”. In this illustration it is possible to select and view segments individually.

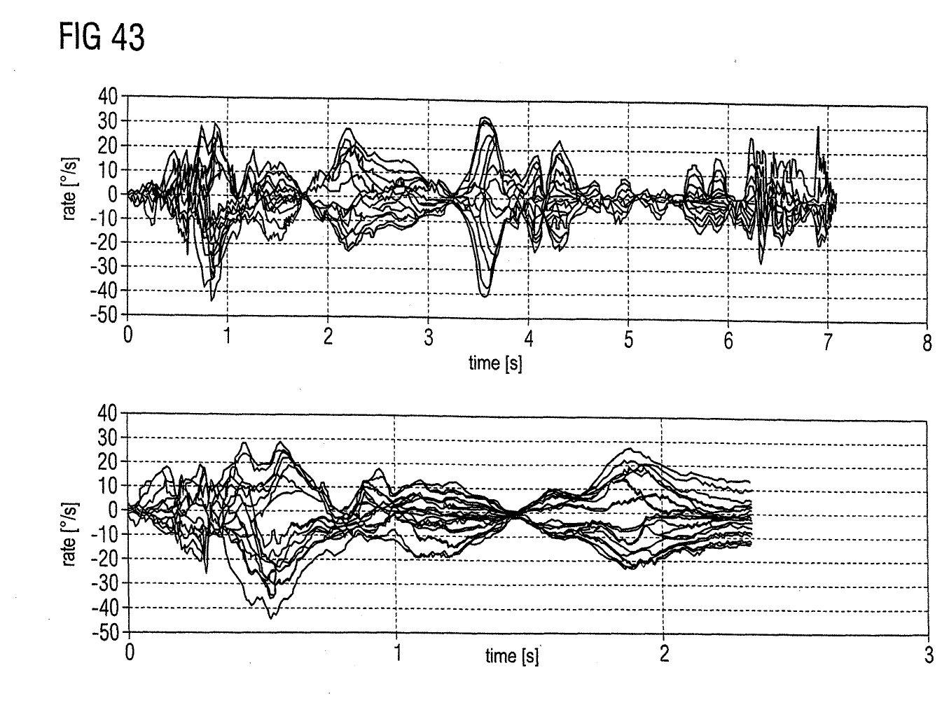

FIG. 43 shows rate illustrations in which the rate is plotted over time (as raw data). It is possible to select and view individual segments.

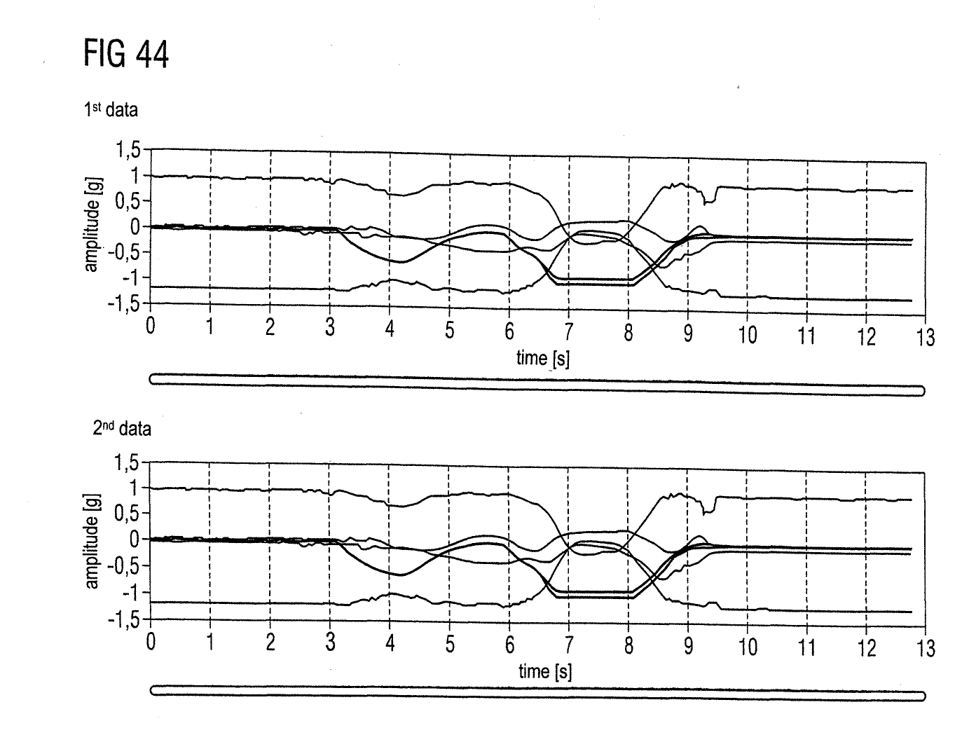

FIG. 44 shows illustrations of the position sensors in which the measurement results of the position sensors are illustrated. All three space axes for the right and left sensors are illustrated over time. The axes are classified in the multiple of the gravitational acceleration (1 g=9.81 ms⁻²).

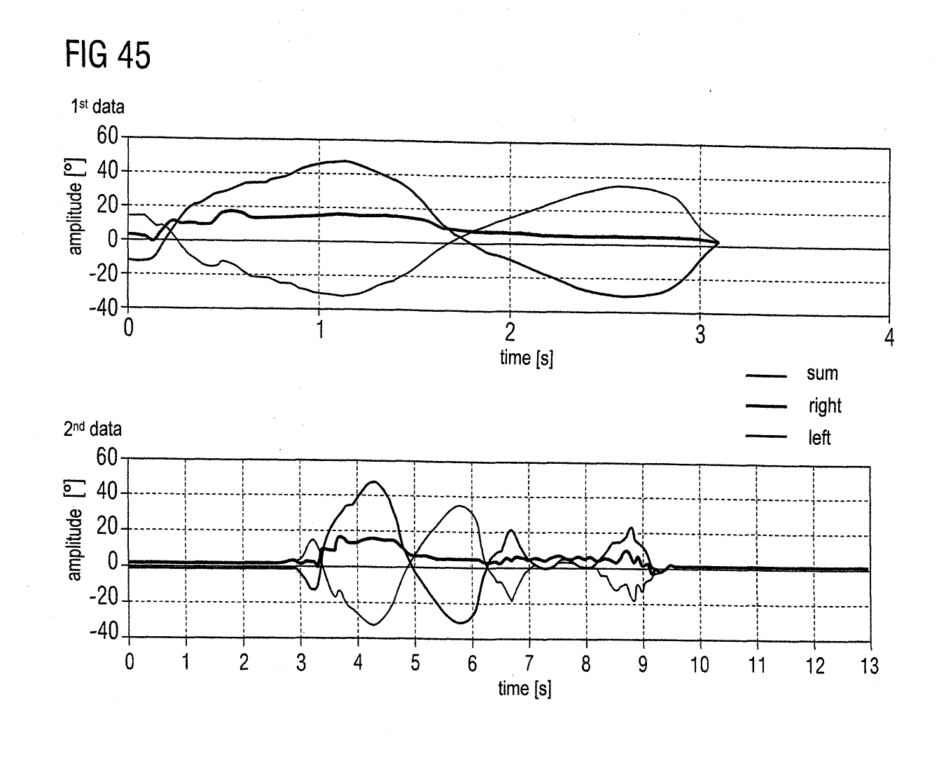

FIG. 45 shows a comparative illustration between the right and the left sensors in which the sensor segments of the right or left sensor, respectively, are each added and are illustrated with the sum of all sensor segments in a plot over time. Also in this plot it is possible to take individual segments out of the illustration. Care has to be taken that the displayed sensor segments are equal for the right and the left sensors since the displays do not make any sense otherwise.

The activity of the subject is to be visualized and evaluated. For this purpose, the following steps and reflections are useful:
Selection of the data to be analyzed.
Are individual segments to be added or not?
Selection of levels or thresholds with suitable distance.
Determination how often the angle sum or an angle of a segment exceeds the individual levels.
Graphical illustration of the counting.

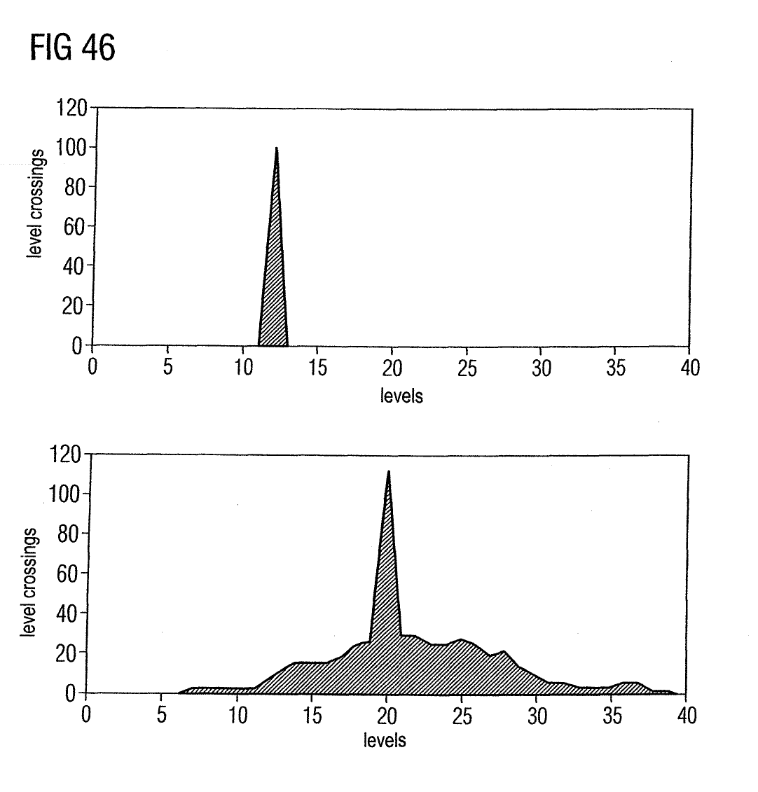

In FIG. 46 two level crossing graphs are illustrated, the one with activity and the other one almost without activity, wherein the number of level crossings for the respective level is illustrated. In the first plot little activity took place. Only few levels were crossed, but they were crossed frequently. The very high, narrow peak results from this fact. In the second plot a phase with more activity took place in addition to the rest phase. The rest phase is represented by the peak, i.e. the high, narrow peak. During the activity phase distinctly more levels are crossed. Due to the increased dynamic of the motion they are, however, crossed distinctly less frequently.

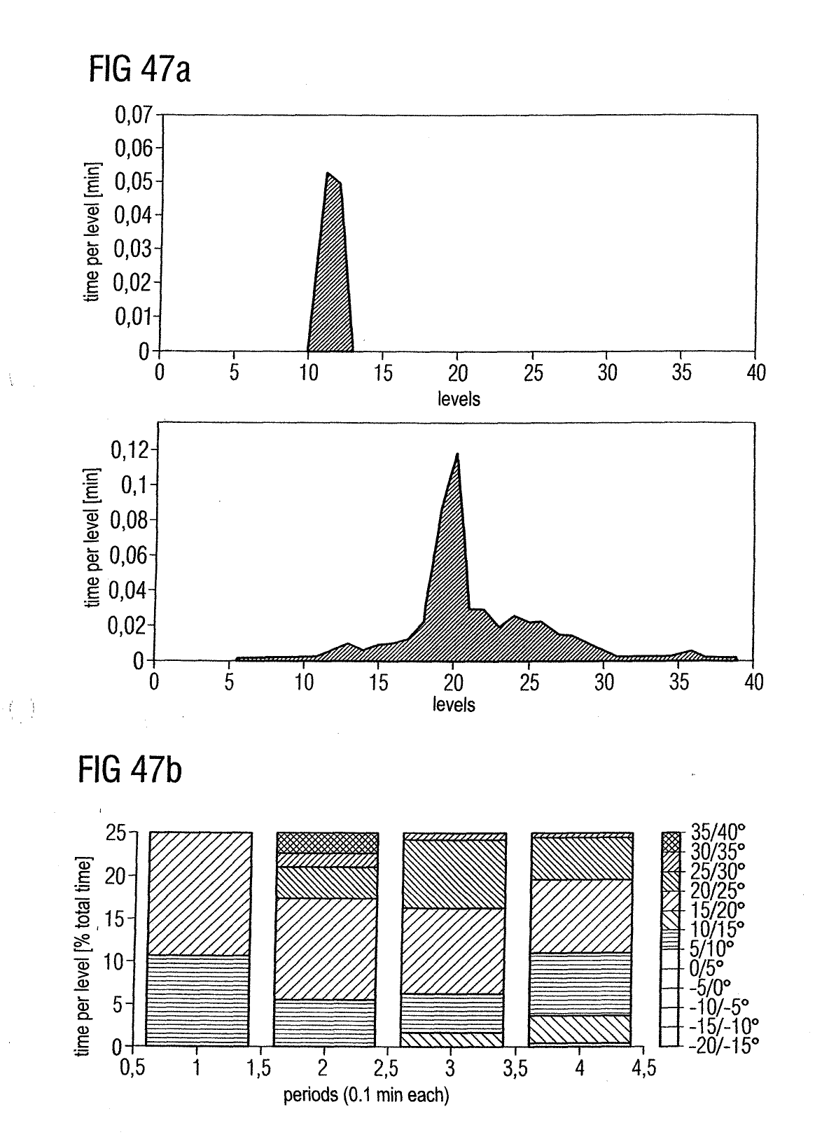

Furthermore, there can be examined how long the angle sum or the angle of the individual segment stayed in the individual intervals between the predetermined levels (for detecting the activity). The “intensity” is thus a measure for how long the subject stays in a particular position. For graphic illustration, the duration (i.e. “dwell time” in a particular position) is plotted for each interval (i.e. for different “positions” of the subject). A regular distribution of several intervals during a time interval indicates high activity. FIGS. 47a and 47b illustrate two different possibilities of graphic illustration for this.

Pain or avoidance motions during a motion are to be detected. For this purpose the evenness of the motion is quantified. An interruption during a motion indicates a pain point, rapid jerks indicate an avoidance motion. The following parameters for the quantification of the evenness of a motion result from this:
Homogeneity of the rate
Homogeneity of the angle
Roundness of the dynamic plot

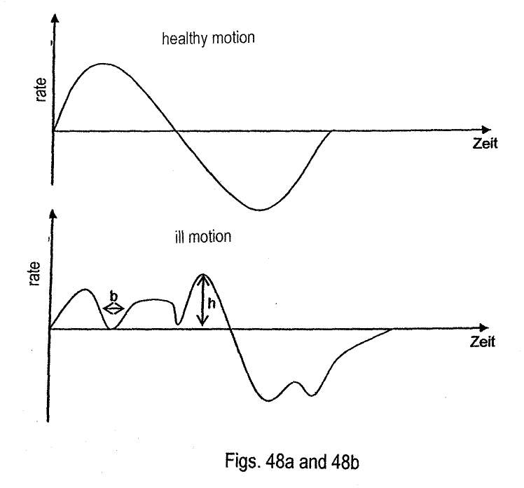

A motion is defined as the distance of the angle amplitude from the initial state up to the return to the initial state. For determining the evenness a slight smoothing of the raw data is additionally useful. FIGS. 48 and 48b illustrate two rate time diagrams with an example of a “healthy” and an “ill motion”.

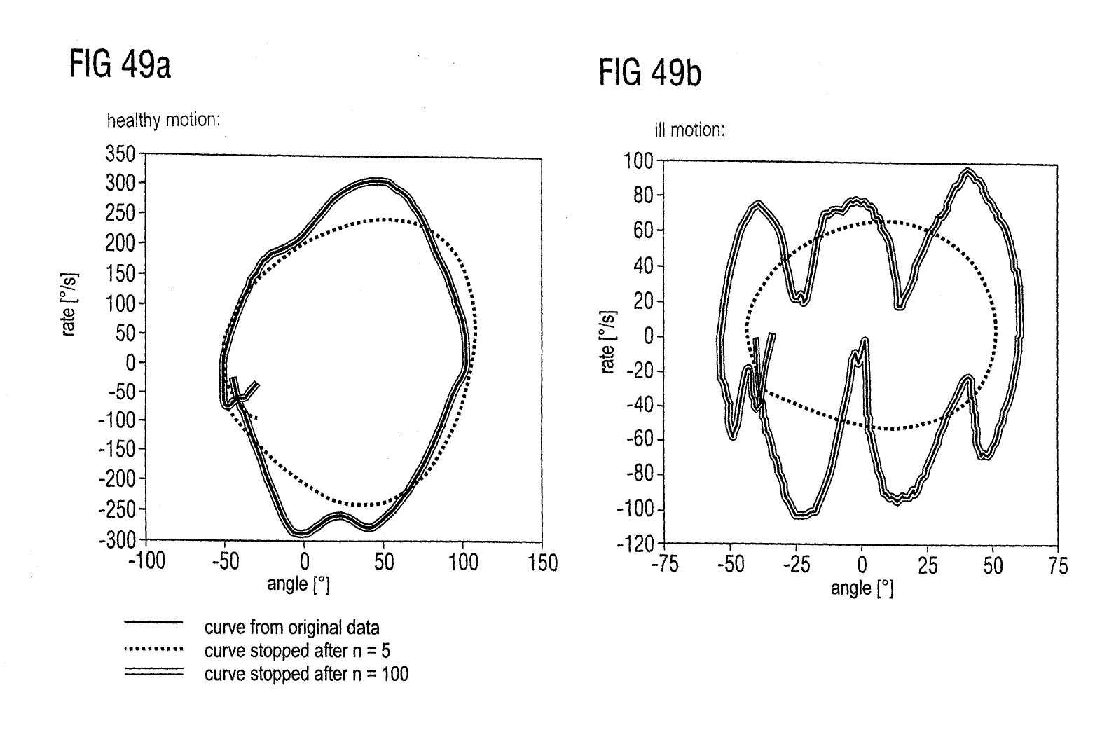

Another possibility of visualization for the evenness of a motion is a phase diagram or dynamic plot as it is illustrated in FIGS. 49a and 49b. Patients with a backache move more carefully and choppy. This difference in evenness is distinctly revealed in the phase diagram (dynamic plot). For the evaluation of the dynamic plot, evenness is to be referred to as a quantifiable parameter. A healthy, pain-free motion will have an almost elliptic figure in the dynamic plot as a consequence. In contrast to this, the dynamic plot of an ill person will show a distinctly more complex figure with some drops in rate.

It is an object of the algorithm to assess the differences quantitatively. For this purpose, the Fourier analysis is used. The Fourier analysis fragments a periodic function to an infinite series of sine and cosine oscillations. The formulae for this are generally known as follows:

$$f ( x ) = \frac{{a}_{0}}{2} + {\sum }_{n = 1}^{∞} [ {a}_{n} \cdot cos ( nx ) + {b}_{n} \cdot sin ( nx ) ]$$

$${a}_{o} = \frac{1}{Π} \cdot {\int }_{0}^{2 Π} f ( x ) d x$$

$${a}_{n} = \frac{1}{Π} \cdot {\int }_{0}^{2 Π} f ( x ) \cdot cos ( nx ) d x$$

$${b}_{n} = \frac{1}{Π} \cdot {\int }_{0}^{2 Π} f ( x ) \cdot sin ( nx ) d x$$

This series expansion is applied both to the angle amplitude and to the angle rate. The further the series expansion is continued, the better the underlying function is approximated.

It is an object to form and compare two approximations: One series expansion that was stopped early (n=2 to 5) and one expansion that approximates the curve very well (n=100 or greater). With the elliptic figures of the healthy motion patterns the series expansion that was stopped early will already form a good approximation. With the more complex curves of ill motions this is not the case. This fact is to be made use of.

From the series calculated for the amplitude and the rates phase diagrams (see FIGS. 49a and 49b) are again formed. Both calculated contours (n=5 and n=100) are to be superimposed as good as possible. The remaining deviation serves as a parameter of evenness. A great deviation means an uneven motion while a small deviation means a healthy motion pattern. Alternatively to the surface area the sum of the error squares after the fitting of the contours (i.e. curve adaptation: adapting the contours to mathematical functions) may also be evaluated as a parameter.

During a long-time measurement for 24 hours and more, very large amounts of data accrue. The processing thereof is very time-consuming. At present, only data amounts of approx. 1 hour can be processed within adequate time. Also algorithms for the calculation of different parameters run too slowly with large amounts of data.

One solution of this problem is a reduction of the amount of data without restricting the parameters relevant for the subsequent analysis, e.g. motion or dynamics. This is to be effected advantageously during the extraction of the data from the memory card. Instead of a constant sampling rate of 100 Hz, a dynamic sampling rate is aspired. The dynamic sampling rate is to be low in the periods in which no motion takes place, and is to be high as soon as the subject is moving.

This can be achieved in that the current measurement value is compared with the last stored value. If the change exceeds a predefined threshold value (e.g. 0.25°), the new value is stored. If the threshold value is not exceeded, the new value is discarded. With the next value the procedure starts anew.

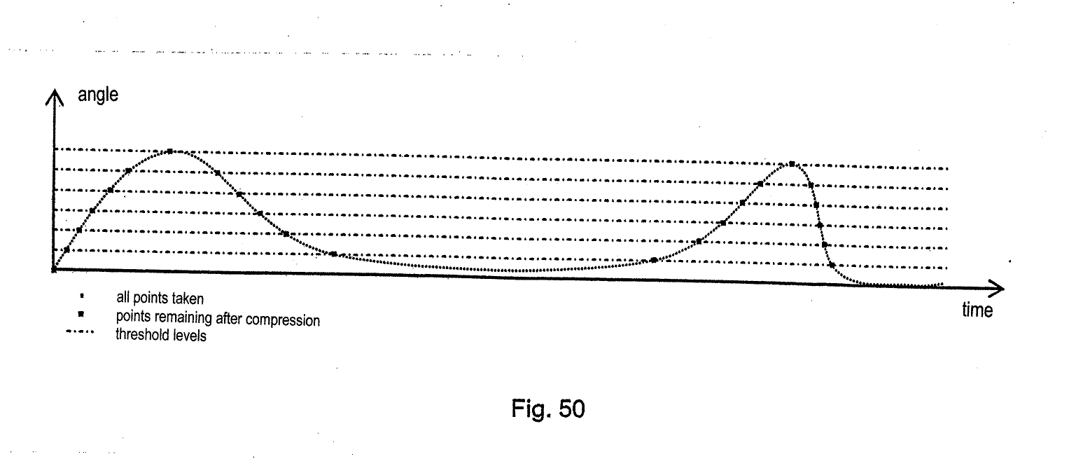

This process is illustrated in FIG. 50. FIG. 50 shows an angle time diagram of an exemplary motion. The individual (small) points of the curve constitute the total number of the recorded data. If this curve crosses one of the threshold levels (dash and dot lines), the predefined threshold value is exceeded and the currently detected data value is stored (illustrated as a fatter point). The fatter points thus symbolize the data remaining after the compression which are used for a subsequent analysis. Since no more statistic recording frequency exists with this method, it becomes necessary that the point in time of the occurrence is stored in addition to the angle.

First tests with large amounts of measured data show that a reduction of the data by a factor 20 is possible with this method without losing dynamic information (in fact, a factor 40 would be possible, but the additionally required time information halves this factor). In addition to the reduction of the amount of data to be processed, the compression factor renders it possible to generally assess the activity of a subject during a long-term measurement. In the case of high activity or a lot of motion, respectively, the compression factor becomes smaller since the threshold values are exceeded more frequently. In the case of little activity, e.g. during sleeping, a distinctly larger compression factor has to be expected.

For some therapeutic decisions the mobility of individual vertebra segments is of great importance. For this purpose, however, a preparation of the detected raw data is necessary.

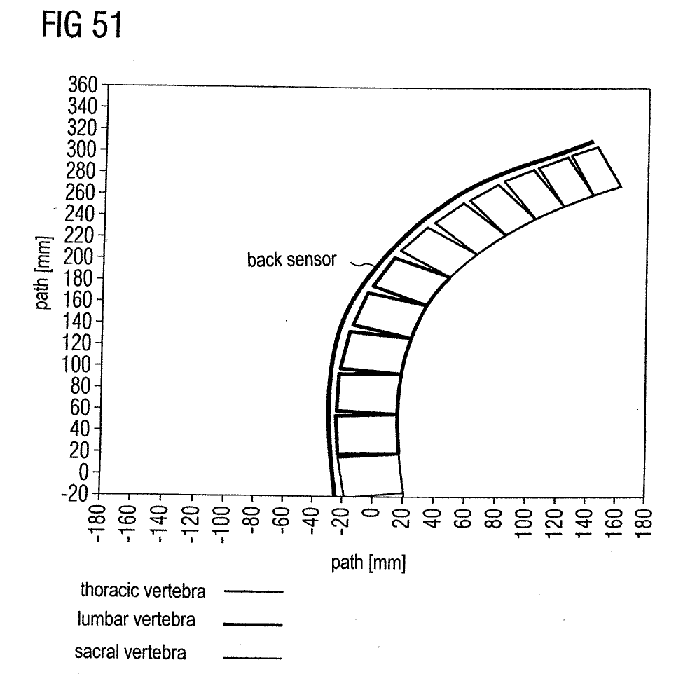

At the beginning of the measurement the distance between the last cervical vertebra C7 and the first sacral vertebra S1 is measured. By means of constant factors (i.e. assuming that all vertebrae between the vertebra C7 and the vertebra S1 have equal size) the size of each vertebra is calculated from this distance. Since the sensor band has a restricted length, only that number of vertebrae that is detected by the sensor depending on the tallness of the patient is illustrated (see FIG. 51).

The direction of the first sensor is determined by the position sensor in the sensor band electronics. The angles between the segments are calculated from the bending data of the sensor band. For this purpose there is determined whether the vertebra is detected only by one or by several segments. In the case of only one segment the vertebra obtains the bending of the segment. If the vertebra is detected over several segments, the calculation of its angle is performed proportionately:

$$angle of vertebra = \frac{\begin{matrix}( length in segment 1 \cdot angle segment 1 ) + \\ ( length in segment 2 \cdot angle segment 2 )\end{matrix}}{total length of vertebra}$$

If more than two segments should span the vertebra, the formula will have to be extended. If the breadth of the vertebra is known, the position of the vertebrae can be calculated more exactly from geometric reflections subsequently.

1. A system for detecting function parameters for the characterization of motion sequences at the human body, comprising:
a bending sensor with at least one strain gauge for detecting bending parameters, wherein said at least one strain gauge changes its impedance during an extension or compression, and sensor electronics for reading out and processing the bending parameters detected by said at least one strain gauge; and
a fixing element for fixing the bending sensor on the skin of the human body, wherein said fixing element is adapted to accommodate said bending sensor such that it is fixed only at one point of said fixing element and follows motions of that part of the human body on which said fixing element is fixed without following possible extensions of the skin of the human body.

2. The system according to claim 1, wherein said bending sensor comprises a plurality of strain gauges which are arranged in a plurality of measuring zones.

3. The system according to claim 2, wherein every two strain gauges are arranged in a measuring zone From said plurality of measuring zones, and wherein said sensor electronics further comprise a Wheatstone measuring bridge and a plurality of toggles that are connected with the measuring bridge, and wherein for each measuring zone from said plurality of measuring zones at least one of said plurality of toggles is provided and is connected with said two strain gauges of the corresponding measuring zone.

4. The system according to claim 3, wherein said toggles are adapted to connect said respective two strain gauges of a measuring zone from said plurality of measuring zones with said measuring bridge, and wherein said sensor electronics further comprise a microprocessor adapted to control said measuring bridge and said plurality of toggles such that always only said two strain gauges of a measuring zone from said plurality of measuring zones are connected with said measuring bridge and the bending parameters detected by said strain gauges are read out sequentially for the different measuring zones.

5. The system according to claim 4, wherein said microprocessor of said sensor electronics is further adapted to read out the bending parameters detected by said strain gauges at a predetermined sampling rate.

6. The system according to claim 5, wherein said microprocessor of said sensor electronics is further adapted to digitize and store the read-out bending parameters.

7. The system according to claim 1, further comprising:
a position sensor for detecting the position of said bending sensor relative to the gravitation field of the earth or the earth's magnetic field.

8. The system according to claim 1, further comprising a memory unit that is adapted to:
read out the processed bending parameters from said sensor electronics and store them; and
be connected with a PC in a wireless or wired manner and to transmit the stored bending parameters to the PC.

9. A method for detecting function parameters for the characterization of motion sequences at the back of a carrier, comprising:
providing a first and a second bending sensor each comprising at least one strain gauge and sensor electronics;
providing a first and a second fixing element for fixing said first and said second bending sensors on the skin of the carrier;
fixing said first and said second bending sensors on the back of the carrier by means of said first and said second fixing elements, wherein said first and said second bending sensors are arranged at the back in a V-shaped manner;
detecting bending parameters by means of said at least one strain gauge of said first and said second bending sensors; and
reading out and processing the bending parameters detected by said at least one strain gauge of said first and/or said second bending sensors by means of said sensor electronics of said first and/or said second bending sensors.

10. The method according to claim 9, further comprising:
detecting of torsional motions by forming the difference from the processed bending parameters of said first bending sensor and the processed bending parameters of said second bending sensor.

11. The method according to claim 9, further comprising:
providing a memory unit;
reading out the processed bending parameters from the respective sensor electronics of said first and said second bending sensors by means of said memory unit; and
storing the read-out bending parameters in said memory unit.

12. The method according to claim 9, further comprising:
detecting a position of said bending sensor by means of a position sensor, wherein the position is detected relative to the field of gravitation of the earth or to the earth's magnetic field.

13. The method according to claim 9, wherein the bending parameters are detected in a space-resolved way by using a plurality of strain gauges for the detection of bending parameters.

14. Computer-implemented method for the analysis of function parameters for the characterization of motion sequences at the human body, comprising:
receiving of measurement values with bending parameters detected with a plurality of bending-sensitive segments of a first and a second bending sensor at different discrete points in time;
converting the bending parameters to pertinent angles;
forming angle sums for the first bending sensor for the different discrete points in time, wherein for each discrete point in time the sum is formed from the angles detected by the plurality of segments of the first bending sensor at the respective discrete point in time;
forming of angle sums for the second bending sensor for the different discrete points in time, wherein for each discrete point in time the sum is formed from the angles detected by the plurality of segments of the second bending sensor at the respective discrete point in time;
forming the difference from the angle sums of the first and the second bending sensors for the different discrete points in time, wherein for each discrete point in time the difference from the angle sum of the first bending sensor for the respective point in time and the angle sum of the second bending sensor for the respective point in time is formed; and
generating a graphic illustration for at least one of the following data sets:
the angle sums of the first bending sensor for the different discrete points in time;
the angle sums of the second bending sensor for the different discrete points in time;
the difference from the angle sums of the first and of the second bending sensors for the different discrete points in time.

15. Computer program for performing the method according to claim 14.
